(19) United States
(12) Patent Application Publication
(10) Pub. No.: US 2022/0106565 A1
(43) Pub. Date: Apr. 7, 2022

(54) MULTICELL CONJUGATES FOR ACTIVATING ANTIGEN-SPECIFIC T CELL RESPONSES

(71) Applicant: Wisconsin Alumni Research Foundation, Madison, WI (US)

(72) Inventors: Jenny Ellen Gumperz, Madison, WI (US); Dana Carina Baiu, Madison, WI (US)

(21) Appl. No.: 17/495,597

(22) Filed: Oct. 6, 2021

Related U.S. Application Data
(60) Provisional application No. 63/088,056, filed on Oct. 6, 2020.

(51) Int. Cl. C12N 5/0783; C07K 14/705; A61K 35/17

(52) U.S. Cl. CPC C12N 5/0636; C07K 14/70575; C12N 2501/2304; A61K 35/17; C12N 2501/22; C07K 14/70532

(57) ABSTRACT

The present invention provides in vitro derived a multicell conjugate comprising an iNKT cell and a dendritic cell (DC). The invention also provides methods of making the multicell conjugate and methods of using the multicell conjugate and compositions comprising the same to treat one or more conditions associated with an antigen or methods of activating an immune response.

### CROSS RELATED APPLICATIONS

This application claims priority to U.S. Provisional Application No. 63/088,056 filed on Oct. 6, 2020, the contents of which are incorporated by reference in its entirety.

### STATEMENT REGARDING FEDERALLY SPONSORED RESEARCH

This invention was made with government support under Grant Number AI136500 awarded by the National Institutes of Health. The government has certain rights in the invention.

### INTRODUCTION

In many diseases, including cancer and chronic viral infections, the immune system is unable to clear the disease because antigen-specific T lymphocytes are suppressed. Current clinical immunotherapies either focus on interrupting suppressive pathways using checkpoint inhibition, or on delivering genetically modified cytolytic effector cells, such chimeric antigen receptor (CAR-T) cells, to directly kill the cancer cells. CAR-T treatment is cumbersome and time consuming as patient cells must be harvested, genetically modified, and cultured for 2-3 weeks to generate sufficient quantities. Checkpoint inhibitor therapies have shown mixed results in clinical trials and are not antigen specific.

Accordingly, a need in the art persists for simple, quick, and effective immunotherapies.

### SUMMARY

The present invention provides an in vitro cultured multicell conjugate between an invariant natural killer T (iNKT) cell and a dendritic cell (DC). The conjugate is formed in culture after about 30 minutes and is maintained in culture for at least 24 hours (1 day), preferably at least 48 hours (2 days), more preferably at least 96 hours (4 days).

In another aspect, the disclosure provides a composition comprising the multicell conjugate described herein and a pharmaceutically acceptable carrier.

In another aspect, the disclosure provides a method of producing a multicell conjugate between an invariant natural killer (iNK) T cell and a dendritic cell (DC), the method comprising: co-culturing an iNKT cell and a DC for a sufficient amount of time to form a stable multicell conjugate.

In a further aspect, the disclosure provides a method of treating a subject with a condition associated with an antigen, the method comprising administering an effective amount of the multicell conjugate or composition described herein to treat the condition.

In another aspect, the disclosure provides a kit comprising the multicell conjugate described herein and instructions for use in treating a disease or condition associated with an antigen.

In a further aspect, the disclosure provides a kit comprising frozen iNKT cells and instructions for producing a multicell conjugate.

In another aspect, the disclosure provides a stable in vitro-derived multicell conjugate between an invariant natural killer T (iNKT) cell and a dendritic cell (DC), preferably wherein the conjugate is maintained in culture for at least 30 minutes.

In a further aspect, the method provides a method of activating T cells in a subject in need thereof, the method comprising administering an effective amount of the multicell conjugate or the composition described herein to the subject, wherein T cell are activated. In some aspects, the T cells are CD8⁺ T cells.

In another aspect, the disclosure provides a method of activating an immune response against an antigen within a subject, the method comprising: administering an effective amount of the multicell conjugate or the composition described herein to the subject, wherein an immune response is activated against the antigen in the subject.

Other embodiments and aspects are described herein.

### BRIEF DESCRIPTION OF THE DRAWINGS

FIG. 1. The combination of iNKT cells and DCs promotes antigen-dependent activation of human T cells. Peripheral blood samples were drawn from healthy adult subjects. Isolated monocytes were cultured in vitro to produce DCs as noted in the Examples. T cells that were autologous to the DCs were isolated from the same samples. CD4⁺ iNKT cells were generated as noted in the Examples, and were usually produced from donors that were allogeneic to the DCs. DCs were co-incubated with a 1:1 ratio of iNKT cells to allow the formation of conjugates, or were cultured alone. Cultures containing DCs were co-incubated with antigens, including Epstein-Barr virus (EBV Ag), Toxic Shock Syndrome Toxin superantigen (TSST SAg), or tetanus toxoid antigen (TT Ag), or were mock-treated (No Ag), and were then co-cultured with T cells that were autologous to the DCs, such that the DCs comprised 2% of the total cells in the culture. In cases where T cell proliferation was measured, the T cells were pre-labeled with a fluorescent dye (e.g. cell trace violet) to allow analysis of the percentage of the T cell population that underwent cell division. (A) Plots showing increased proliferative responses over time of CD8⁺ (left) and CD4⁺ (right) T cells in response to the combination of iNKT cells and DCs loaded with EBV antigens. There was no detectable T cell proliferation in response to isolated iNKT cells and EBV antigen. (B) Plots showing increased proliferative responses over time of CD8⁺ (left) and CD4⁺ (right) T cells in response to the combination of iNKT cells and DCs loaded with TSST superantigen. Plots show the responses of T cells bearing VI32⁺ T cell receptors, which is the subset that is specifically activated by the TSST superantigen. (C) Plot showing frequency of CD4⁺ T cells producing the cytokine interferon-γ (IFN-γ) after 24 hours of exposure to DCs alone, iNKT cell and DC mixture, or lipopolysaccharide-activated DCs alone (+LPS). Each symbol represents an independent T cell activation experiment. iNKT cells were allogeneic to T cells and DCs. (D) Plot showing that only low frequencies of iNKT cells are required to promote T cell activation. T cells were incubated with autologous DCs comprising 2% of the culture, and iNKT cells were added at the indicated frequencies, and the frequency of IFN-γ producing T cells was analyzed after 24 hours. (E) Results showing that cell contact is required for the T cell-activating effects of iNKT cells+DCs. T cells and autologous DCs were co-cultured with iNKT cells either together in a well (lower well, contact condition), or T cells and autologous DCs were exposed to iNKT cells and DCs that were separated by a transwell membrane (upper well, exposure only to secreted factors). Amounts of IFN-γ secreted into the culture were determined by ELISA.

FIG. 2. Cultured human iNKT cells and monocyte-derived DCs form tightly adhered conjugates. Peripheral blood samples were drawn from healthy adult subjects. Isolated monocytes were cultured to produce monocyte-derived dendritic cells (DCs). CD4⁺ iNKT cells were isolated from peripheral blood and expanded in culture. In most cases, iNKT cells and DCs were from unrelated blood donors (i.e. allogeneic to each other). (A) Detection of adhered cells in iNKT-DC co-cultures. iNKT cells and DCs were mixed at a 1:1 ratio and cultured together for 24 hours. Flow cytometric analysis of cells from iNKT+DC co-cultures revealed the presence within the culture of adhered cells (lower left plot, showing a population with distinct light-scatter properties), and analysis of the markers expressed by DCs (DC-SIGN) or iNKT cells (CD5) indicated that DCs and iNKT cells had formed conjugates (lower right plot). (B) Demonstration of heterologous adherence. iNKT cells were labeled intracellularly with a violet fluorescent dye (CTV) and DCs were intracellularly labeled with a fluorescein-derived dye (CFSE). The fluorescently labeled iNKT cells and DCs were mixed together at a 1:1 ratio and incubated in culture medium for 24 hours, then the cells were vigorously resuspended in buffer containing a calcium chelating agent (to break apart weakly associated cells). Plot shows results of flow cytometric analysis of iNKT+DC co-culture (right panel) compared to DCs cultured alone (middle panel) or iNKT cells cultured alone (left panel). Boxed events on the right plot have both fluorescent colors and thus represent tightly associated conjugates of iNKT cells and DCs. (C) Plot showing that nearly all of the tightly adhered cells are comprised of one DC and one iNKT cell. iNKT cells and DCs were mixed at a 1:1 ratio and co-cultured for 24 hours. Plot shows percent of the iNKT+DC conjugates in 3 replicate co-cultures containing the indicated iNKT cell and DC compositions, as assessed by imaging flow cytometry. (D) Demonstration that the frequency of conjugates within the culture can be modulated by added factors. DCs were cultured in a standard culture medium containing bovine serum, or were pre-exposed to human serum albumin (HSA) containing bound lipids, and then co-incubated at a 1:1 ratio with iNKT cells. Plots show results of flow cytometric analysis indicating that the frequency of iNKT-DC conjugates is increased when DCs are pre-treated with HSA. (E) Demonstration that iNKT cells form conjugates with DCs generated from induced pluripotent stem cells (iPSCs). iPSCs were re-differentiated into a myelo-monocytic lineage and then further differentiated to produce DCs. The iPSC-derived DCs were cultured for 24 hours with a 1:1 ratio of iNKT cells and the frequency of stably adhered cells was assessed by flow cytometry.

FIG. 3. Durability of iNKT-DC conjugates in vitro and in vivo. (A) iNKT cells were labeled intracellularly with a violet fluorescent dye (CTV) and DCs were intracellularly labeled with a fluorescein-derived dye (CFSE). The fluorescently labeled iNKT cells and DCs were mixed together at a 1:1 ratio and incubated in culture medium for the indicated times, then the frequency of iNKT-conjugated DCs was determined by flow cytometry. Plot shows that the percent of DCs conjugated with iNKT cells is maintained in vitro for at least 96 hours. (B) Fluorescence microscopic imaging of iNKT+DC co-cultures showing conjugates of iNKT cells (red) and DCs (green) after the indicated amounts of incubation time. (C) Demonstration that adhered pairs of human iNKT cells and DCs can be detected in vivo after administration to immunodeficient mice. DCs labeled with CTV (red color) and iNKT cells labeled with CFSE (green color) were cultured for 24 hours to allow the formation of conjugates, then the mixture was intravenously injected into an immunodeficient mouse strain (NSG). Tissues were collected 24 hours after injection, and analyzed by fluorescence microscopy for evidence of adhered iNKT-DC pairs. Panels show sections of murine lung tissue containing DCs closely associated with iNKT cells (yellow arrows). (D) Quantitation of isolated iNKT cells, isolated DCs, and iNKT-DC conjugates detected in murine spleen, lung, and liver tissue collected at 24 hours post-injection.

FIG. 4. Upregulated expression of co-stimulatory molecules by iNKT+DC conjugates. DCs were co-cultured with iNKT cells, or with the synthetic adjuvant GLA (Glucopyranosyl Lipid Adjuvant), or were mock-treated, and then flow cytometric staining was performed to detect cell surface expression of co-stimulatory molecules or antigen presenting molecules. (A) Plot showing aggregated results from independent experiments where surface expression of the indicated molecules on iNKT+DC conjugates was normalized by the respective expression levels of mock-treated DCs (i.e., up-regulation of co-stimulatory markers and antigen presenting molecules induced by conjugation). All of the markers shown are significantly upregulated on iNKT+DC conjugates compared to mock-treated DCs. (B) Plot showing aggregated results from independent experiments where surface expression of the indicated molecules on iNKT+DC conjugates after 24 hours of co-culture was normalized by those of GLA-treated DCs (i.e., comparison of conjugation effect to that of a synthetic adjuvant). While conventional co-stimulatory molecules (CD80, CD86, CD83) and class II MHC molecules (HLA-DR) are expressed at similar levels on iNKT+DC conjugates as GLA-treated DCs, class I MHC (HLA-ABC) molecules and a series of unusual co-stimulatory molecules are expressed at significantly higher levels on iNKT+DC conjugates than on GLA-treated DCs. (C) Plot showing that the level of expression of PD-L1 (an inhibitory molecule, aka a “checkpoint” ligand) is about 50% less on iNKT+DC conjugates than on GLA-treated DCs. (D) Culture supernatants from DCs alone, iNKT+DC co-cultures, or iNKT cells alone were tested for concentrations of IL-12p70 (a cytokine known to promote a TH1 response by T cells), or IFN-γ (a TH1 cytokine). Results show that both cytokines are secreted in iNKT-DC co-cultures.

FIG. 5. iNKT cells and DCs each contribute to the co-stimulatory profile of the conjugates. (A) Imaging flow cytometric analysis of fluorescently stained conjugated cells from iNKT+DC co-cultures analyzed in an ImageStream instrument indicated the markers expressed by iNKT cells and DCs. iNKT cells, but not DCs, expressed CD70, whereas DCs, but not iNKT cells, expressed other co-stimulatory molecules. (B) Plot showing levels of CD70 expression on iNKT cells from freshly isolated human spleen (primary iNKT cells) compared to the levels on iNKT cells that were expanded in culture for 2-3 weeks (cultured iNKT cells) or to iNKT cells that were conjugated to DCs (conjugated iNKT cells). Compared to primary iNKT cells, CD70 is significantly upregulated on cultured iNKT cells, and in some cases appears to become further upregulated when iNKT cells form conjugates with DCs.

FIG. 6. Key co-stimulatory molecules are selectively up-regulated on conjugated cells and the up-regulated expression is maintained for at least 96 hours in vitro. (A) Comparison of extent to which the indicated markers are up-regulated on iNKT+DC conjugates relative to those of unconjugated DCs in the same culture. Conventional co-stimulatory molecules (CD80, CD86, CD83) are only modestly increased on conjugates, whereas a series of tightly regulated co-stimulatory molecules (CD70, 4-1BBL, OX40L, and IL-15Rα) are substantially up-regulated on conjugates. (B) Plot showing that up-regulated expression of key co-stimulatory molecules is maintained on iNKT+DC conjugates for at least 96 hours in vitro.

FIG. 7. Anti-tumor effects of iNKT+DC mixture. The ability of iNKT cells+DCs to promote anti-tumor responses by human T lymphocytes was tested using a humanized mouse lymphoma model. Human umbilical cord blood mononuclear cells (including B-lymphocytes, T lymphocytes, monocytes, and DCs) are briefly exposed to Epstein-Barr Virus (EBV, a human B cell-specific γ-herpes virus that drives formation of B cell lymphomas). The EBV-exposed cells are injected intraperitoneally into immunodeficient mice. This leads to the formation of human B cell lymphomas in the peritoneal cavity within 3 weeks. Tumors typically invade organs such as pancreas, liver, bile ducts. Similar to many human cancers, the T cells become suppressed and fail to kill the tumors. (A) iNKT cells+DCs promote specific killing by T lymphocytes. Human T cells were removed after 28 days from mice that had developed tumors (left plot) or from mice that were injected with uninfected cord blood mononuclear cells and did not have tumors (naive to EBV, right plot). The T cells were mixed with autologous DCs or with autologous DCs and allogeneic iNKT cells and tested in vitro for killing of autologous target cells, using an IncuCyte live cell imaging instrument. Plots show specific lysis of target cells over time in replicate samples (symbols and error bars represent means and standard deviations). (B) Administering iNKT cells+DCs promotes tumor clearance in vivo. Mice were injected with EBV-treated human umbilical cord blood mononuclear cells to drive the formation of human B cell lymphomas in vivo. After 25 days the mice were intravenously injected with iNKT cells alone, autologous DCs alone, a 1:1 mixture of iNKT cells+autologous DCs, or mock-treated with sterile buffer (vehicle). Six days later the mice were euthanized and all visible tumor tissue was excised from the peritoneal cavity. Plot shows tumor mass, with each symbol representing the results from an individual mouse. (C) Histological sections of pancreas tissue from human EBV-driven lymphoma model mice. Left image shows tissue from a vehicle-treated mouse, with pancreas heavily invaded by lymphoma cells. Right image shows tissue from a mouse given iNKT cells and DCs, where an area of pancreas that was probably previously being invaded by tumor appears to have been cleared of lymphoma cells. (D) Spleen tissue from 3 iNKT+DC-treated and 3 vehicle-treated mice were tested for expression profiles of human immune-related genes. A variety of genes were significantly upregulated (red bar) in mice that received the iNKT+DC mixture, while these were down-regulated in mice that received vehicle. A smaller number of genes were significantly down-regulated (blue bar) in mice that received the iNKT+DC mixture, but were upregulated in mice that received vehicle. In the iNKT+DC upregulated group were multiple genes associated with T cell activation, while the group that was down regulated after iNKT+DC treatment included genes associated with EBV-infected B cells. (E) Mice bearing EBV-driven B cell lymphomas were intravenously injected after 25 days with iNKT cells combined with DCs that were either allogeneic (allo) or autologous (autol) to the EBV-infected B cells and T cells in the mice. Plot shows tumor burden for each individual mouse. NS indicates there is no significant reduction in tumor burden following administration of iNKT cells and allogeneic DCs, whereas administration of iNKT cells and autologous DCs produces a significant (P=0.0025) reduction in tumor burden.

### DETAILED DESCRIPTION

The invention relates to methods for preparing stable and durable multicell conjugates (adhered pairs) of human invariant natural killer T cells (iNKT cells) and dendritic cells (DCs), referred to herein as “multicell conjugates”, “iNTK+DC conjugates” “stable multicell conjugates” or as “the conjugates,” and to preparations thereof. The invention further relates to methods for using the conjugates to activate human T cells and to promote antigen-specific responses and immune responses.

The multicell conjugates described herein are artificially manipulated in vitro to form cell-cell adhered pairs of iNKT and DC cells. The methods described herein demonstrate that multicell conjugates are formed in vitro tissue culture after about 30 minutes in culture, and the addition of human serum albumin (HSA) containing lysophospholipids to the culture condition increases the number of conjugates, which allows for an increase in the number of conjugates formed in vitro. Not to be bound by any theory, but the ability to form more conjugates in vitro can contribute to the potency of multicell conjugates used for in vivo applications, reducing the number of cells required for effective therapy. As further demonstrated in the examples, these conjugates are maintained in culture for at least 24 hours, and have been shown to still be adhered after at least 96 hours in culture. Further, the Example demonstrates that these multicell conjugates are able to remain adhered after in vivo administration, and were demonstrated to be found in vivo at least 24 hours after administration. These multicell conjugates can traffic in vivo from the site of administration to active sites within the subject.

The DCs in the multicell conjugates express one or more co-stimulatory molecules and HLA as described herein. The iNKT cells in the multicell conjugates express the CD70 co-stimulatory molecule, which is not typically found on primary iNKT cells. The combination of iNKT cells and DCs produces stimulatory cytokines, and thus constitutes an in vitro engineered complex that provides multiple powerful activating signals to T cells.

The stable multicell conjugates described herein have been developed to have durable tight-adherence, both in vitro and in vivo. The term “stable” is used interchangeable with the terms “tightly adhered,” “tight adherence,” and “durable” with respect to the conjugates and refers to the ability of the iNKT-DC conjugates to remain adhered to each other for a period of time (e.g., in in vitro culture). The conjugates remain stably associated (or tightly adhered) after being exposed to a calcium and magnesium-free phosphate buffered saline (PBS) buffer containing 2 mM EDTA for 10 minutes to 1 hour, and/or being subjected to shear forces associated with vigorous pipetting and centrifugation. The stable conjugates are further able to be maintained as an adhered conjugate both in in vitro and in vivo. The conjugates are able to remain adhered to each other over 24-96 hours in in vitro culture, and for at least 24 hours after in vivo administration. Thus the term stable encompasses the ability of the conjugate to be maintained as a conjugate for at least 24-96 hours. The multicell conjugate activates antigen-dependent CD8⁺ T cells (often called cytotoxic T cells) and CD4⁺ T cells (often called helper T cells), as demonstrated in the examples. Therefore, the present disclosure provides methods of producing and compositions comprising these newly developed iNKT-DC multicell conjugates.

To make the preparations, human iNKT cells are combined with human dendritic cells (DCs) in vitro. To facilitate the ability of the iNKT+DC conjugate to elicit an antigen-specific immune response upon contact with a T cell (for example, after administration to the subject), the DC may be exposed to, and loaded with, an antigen before, during or after conjugation with the iNKT cell, as detailed in the methods below. In one embodiment, the DC is contacted with the antigen in vitro, and the loaded DC (i.e., DC presenting the antigen or fragment thereof) is then cultured with iNKT cells to form the iNKT+DC conjugate. In another embodiment, the DCs are concurrently incubated with the antigen and the iNKT cells to form the iNKT+DC conjugate. In another embodiment, the iNKT+DC conjugate is formed prior to incubation with the antigen whereby the antigen is loaded on the DCs within the iNKT+DC conjugate. These different methods allow for the specific antigen loading of the iNKT+DC cells with known antigens. This antigen-loaded iNKT+DC conjugate is able, if administered to a subject, to elicit an antigen-specific immune response within the subject, particularly an antigen-specific T cell response. iNKT cells, also known as type I or classical NKT cells, can be isolated from blood of healthy subjects and expanded in tissue culture using available methods detailed below. The DCs can be generated in tissue culture by differentiating freshly isolated monocytes using available methods, detailed below, or by differentiating from pluripotent stem cells or hematopoietic progenitor cells. In some embodiments, the iNKT+DC conjugated cells can further be selected or separated from unconjugated cells in the preparation to obtain a pure multicell conjugate preparation. In some embodiments, a selection or separation step is not necessary, as there are sufficient conjugates formed in the in vitro culture for use and administration to a subject without a purification step, as described herein. These multicell conjugate preparations elicit an antigen-specific immune response that may be used to treat a condition associated with an antigen, including but not limited to cancer, pathogen infection, among others.

In another embodiment contemplated, the iNKT+DC conjugate may be exposed in vivo to the antigen, e.g., local administration of the iNKT+DC conjugate to a site containing antigens (e.g., inside a tumor, bone marrow, intradermal, infection site, etc.). This allows for uptake of antigenic materials in situ by iNKT+DC conjugates so as to stimulate an immune response specific to the in vivo derived antigens.

On their surface, the multicell conjugates express various ligands that are required to efficiently activate T lymphocytes, including the CD80 and CD86 co-stimulatory molecules and MEW molecules, and these ligands are present at levels higher than on the surface of DCs that have not been activated. Additionally, the conjugates show higher expression of the co-stimulatory ligands CD70 (on the iNKT cell) and 4-1BBL (CD137L), OX40L (CD134L), and IL-15 receptor alpha chain (CD215), or combination thereof and lower expression of the inhibitory ligand PD-L1 than unconjugated DCs treated with a synthetic adjuvant (glucopuranosyl lipid adjuvant, GLA). Furthermore, iNKT+DC conjugates activate antigen-specific human T cells in a contact-dependent manner. This antigen-specific T cell activation treats conditions associated with an antigen, for example, to reduce, inhibit or eliminate tumor cells in a subject. As demonstrated in the examples, administration of the conjugates in a humanized mouse model of Epstein-Barr virus (EBV) induced B cell lymphoma resulted in clearance of tumor masses, and exposure to iNKT+DC conjugates selectively caused T cells from mice bearing EBV-induced lymphomas to kill target cells in vitro.

The multicell conjugates as described herein and produced by the methods herein may have one or more of the following characteristics. The iNKT and DC cells in the multicell conjugate remain tightly associated for at least 24 hours, preferably at least two days (48 hours), more preferably for at least 4 days (96 hours) in vitro after formation. The multicell conjugates preferably comprise between two and three cells, and preferably comprise one iNKT cell and one DC. The multicell conjugate described herein maintains expression of one or more co-stimulatory molecules on the conjugates for at least two (2) days, preferably at least four (4) days in vitro. The multicell conjugate comprises an iNKT cells which expresses high levels of CD70, and DCs expressing one or more of the other co-stimulatory molecules/ligands described herein. These iNKT cells used to generated conjugates differ from primary iNKT cells, which express little or no CD70 on their cell surface. This combination of CD70 expression and one or more co-stimulatory molecules (CD215, CD137, CD252, CD80, CD86, etc.) on the multicell conjugates described herein are not found on cells isolated from a subject and are unique to the multicell conjugates of the present invention. The conjugates are able to be formed by coculture of iNKT cells and DC cells in culture for around 30 minutes or more and then remain tightly associated for at least 24 hours, more preferably at least 96 hours as described herein.

iNKT cells used in the multicell conjugate are suitable for both allogeneic and autologous immunotherapeutic use, as they recognize CD1d, a non-polymorphic antigen-presenting molecule. Because iNKT cells can be frozen and stored for later use, the invention also provides a novel cellular immunotherapy wherein conjugation-ready “off the shelf” allogeneic iNKT cells are provided for conjugation to DCs generated as needed from monocytes or other cells derived from an individual subject in need of treatment. Alternatively, the iNKT cells may be conjugated to DCs derived from induced pluripotent stem cells (iPSCs) that are chosen or engineered so as to express specific MHC types (i.e. HLA types), thus allowing for a fully “off-the-shelf” reagent that does not require biological material collected from individual subjects. iNKT+DC conjugates thus prepared can be loaded with one or more antigens (e.g., tumor cell antigens or pathogenic antigens) and administered as a cellular immunotherapy to activate the patient's T cells as detailed below.

The present invention offers advantages over current immunotherapies. (1) The invention activates T cells, whereas checkpoint-inhibition strategies interrupt T cell suppressive pathways; accordingly, the two approaches are expected to act synergistically. (2) The mode of therapeutic action of the invention is antigen-specific, whereas checkpoint inhibition strategies are not; accordingly, this strategy is expected to be less likely to result in pathology arising from inappropriate T cell activation. (3) The invention does not require genetic modification of a recipient's cells, as is required when generating CAR-T cells. (4) iNKT+DC conjugates of the invention can be prepared in just a few days, much faster than the several weeks typically required to generate therapeutic quantities of CAR-T cells. (5) The invention requires comparatively few primary cells from the subject because antigen-loaded DCs are highly efficient T cell activators.

### Methods of Producing a Multicell Conjugate

The present disclosure provides methods for producing a multicell conjugate between an iNKT cell and a DC, the methods comprising: co-culturing an iNKT cell and a DC in selectively formulated culture medium for a time sufficient to form a stable multicell conjugate. iNKT cells are mixed with DCs in culture (e.g., 37° C., 5% CO₂) for at least 15 minutes, preferably at least 30 minutes, alternatively at least one hour, and can be left in culture for two days (48 hours) or more, alternatively four days (96 hours) or more. The iNKT cell and DC are co-cultured at suitable ratios to produce multicell conjugates, for example, an iNKT:DC ratio between 5:1 and 1:1. Suitably, the iNKT cell used in the method is a substantially pure (e.g., greater than 95%) population of CD4⁺ iNKT cells as determined by expression of an appropriately rearranged T cell receptor as identified through binding of recombinant CD1d molecules loaded with lipid antigen (α-galactosylceramide or one of various related chemical compounds) or by the binding of a specific monoclonal antibody (e.g. clone 6B11 (commercially available at BioLegend, see also, e.g., Rout N, et al. 2010. PLoS One 5:e9787. (FC)) and co-expression of the CD4 co-receptor.

In some embodiments, in the iNKT cell population, greater than 95%, 98%, 99% of the cells are iNKT cells, preferably greater than 99% iNKT cells, including 100%. Suitable iNKT cell populations are discussed below.

Suitably, the DCs used in the method are also a substantially pure population (greater than 95%) of DCs. Characterization of DCs can include expression of MHC class I or MHC class II molecules, expression of one or more DC marker or co-stimulatory molecule found on DCs (e.g., CD209, CD86, CD80, etc.), and low expression of markers found on non-DCs, for example, low expression of CD14 (monocyte marker), detailed more below. In some embodiments, the DCs are at least 98% pure, alternatively at least 99% pure, including 100% pure.

The iNKT-cell conjugates preferably comprise two to three cells, preferably 2 cells, one iNKT cell and one DC.

It is preferable that the iNKT cell and DCs are conjugated in conditions that result in at least 20% of the iNKT cells and DCs forming stable conjugates. In some embodiments, the conjugates may need to be selected from the non-conjugate cells, e.g., by size or other methods described herein.

### Dendritic Cells

DCs are heterogeneous populations of antigen-presenting myeloid and plasmacytoid leukocytes that originate in the bone marrow or in some cases may differentiate from circulating progenitor populations (e.g. monocytes) in vivo. DCs capture and process antigenic proteins, then convert the proteins to peptides for presentation on their surface by MHC molecules, such that the peptide antigens are able to be recognized by T cell receptors (TCRs). This is referred to as loading of the DC with antigens (i.e., presentation of the antigen or fragment thereof on MHC molecules on the surface of the DC).

iNKT+DC conjugates containing DCs presenting one or more specific antigens, can then be used to activate antigen-specific T cells within a subject. Suitable antigens are discussed below. DCs can be derived in vitro by obtaining monocytes from a blood or bone marrow (BM) sample and then differentiating the monocytes to DCs by culturing them with specific cytokines.

Alternatively, dendritic cells can be derived from differentiation of induced pluripotent stem cells (iPSCs) or hematopoietic progenitor cells, by methods known in the art.

Characteristically, the DCs for use in the multicell conjugates described herein express major histocompatibility complex (MHC) class I or MHC class II molecules, in addition to one or more DC marker or co-stimulatory molecules as described herein. Preferably, in one embodiment, the DCs may have increased expression of either or both of MHC class I and MHC class II markers on their surface. For example, the DCs express elevated MHC molecules and one or more markers associated with dendritic cells (e.g., CD209 (DC-SIGN), CD1a, CD1b, or CD1c), have elevated expression of CD80 and/or CD86 co-stimulatory molecules, and little or no expression of markers that are elevated on monocytes and macrophages (e.g., CD14, CD68). Additionally, DCs are negative for markers of other leukocytes (e.g., CD19 (B cell), CD3 (T cell), CD56 (NK cell)). Thus, in some embodiments, the dendritic cells may express one or more of the co-stimulatory markers selected from CD80, CD83, CD86, CD134L (OX40L), CD137L (4-1BBL), CD215 (IL-15Ra) and combinations thereof. Other co-stimulatory molecules expressed on DCs are known in the art and contemplated herein. DCs are known for their slightly larger size than primary lymphocytes and have smooth (not highly granular) cytoplasm. They display multiple protrusions (dendrites) at the cell surface and are only slightly adherent (i.e. not highly adherent and stretched or flattened out on the surface of the tissue culture plate). In some embodiments, the DC express CD209 and low levels of CD14. In some embodiments, the DCs can be monocyte derived and express CD209.

As used herein, the term major histocompatibility complex or MHC refer to molecules found on the surface of DCs which function to display peptide fragments and proteins from within the cell for T-cell activation. In humans, the human leukocyte antigen (HLA) proteins are proteins encoded by the major histocompatibility complex (MHC) gene complex. Therefore, as used herein, the term MHC encompasses HLA molecules (i.e., MHC I encompasses HLA class 1 (HLA A, B, and C) in humans and MHC II encompasses HLA DP, DQ and DR in humans). In some preferred embodiments, the DCs have increased cell surface expression of either or both of MHC class I proteins (e.g., HLA A, B and C proteins on human cells) or MHC class II proteins (e.g., HLA DR, DP, DQ proteins on human cells).

In some embodiments, the DCs are allogenic to the conjugated iNKT cells. In other embodiments, the DCs are autologous to the conjugated iNKT cells. In still other embodiments, the DCs are autologous to the subject in need of treatment and the conjugated iNKT cells are allogeneic to the subject in need of treatment. In yet other embodiments, the DCs and conjugated iNKT cells are both allogeneic to the subject in need of treatment. Depending on the subject to be treated, the DCs may be allogeneic but partially or fully HLA-matched to the treatment subject. Alternatively, if the DCs are iPSC-derived, the DCs may be engineered to express limited HLA-allotypes and selected as to match the treatment subject.

DCs used in the present invention may be derived from monocytes. DC can be obtained from a monocyte by a method comprising culturing in suitable medium for a time adequate for the monocyte to differentiate into a DC. For example, the monocyte may be a CD14⁺ monocyte cultured in medium comprising recombinant human GM-CSF and recombinant IL-4 in an amount sufficient to differentiate the monocytes into DCs. Differentiation of monocytes to DCs can be characterized as known in the art, for example, by loss of expression of CD14 (monocyte marker), gain of expression of one or more DC markers (e.g., CD209, CD1a, CD1b, or CD1c expression, elevation of CD80, CD83 CD86, MHCII) or lack of expression of non-DC markers, for example B-cell marker, T cell marker, etc., or a combination thereof. Suitably, the monocytes may be cultured for at least 2 days in the presence of GM-CSF and IL-4, alternatively for at least 3 days. DCs may be further sorted from the culture by methods known in the art, for example, magnetic bead separation or flow cytometric sorting using anti-DC antibodies (e.g., anti-CD209), anti CD80 or CD86 antibodies, and in some examples, lack of expression of other markers (CD14⁻, CD19⁻, CD3⁻, CD56⁻, etc.). For example, CD209⁺CD14⁻ cells may be isolated from the cultured monocytes.

Monocytes may be obtained by known methods in the art. In one embodiment, the monocytes may be isolated from a blood sample; specifically monocytes can be isolated from peripheral blood mononuclear cells from a blood sample. A peripheral blood mononuclear cell (PBMC) is any peripheral blood cell having a round nucleus, for example, lymphocytes (T cells, B cells, NK cells) and monocytes. PBMCs can be isolated from a blood sample by methods known in the art, for example, by density gradient centrifugation using a hydrophilic polysaccharide such as Ficoll (TM of GE Healthcare), or Lymphoprep (TM of StemCell Technologies) to separate blood samples into layers, specifically a top layer of plasma, followed by a layer of PBMCs and a bottom fraction of polymorphonuclear cells (i.e., neutrophils and eosinophils) and erythrocytes. Monocytes can be further isolated from the PBMCs via methods known in the art, including, but not limited to, for example, rosette centrifugation to remove other cell types, adherence plating (monocytes will adhere to plastic while other lymphocytes will not), and magnetic or fluorescent sorting of cells using monocyte specific antibodies (e.g., using anti-CD14 antibodies). In some embodiments, CD14⁺ monocytes are isolated from PBMCs via magnetic sorting or flow cytometry. Monocytes may also be isolated via flow cytometry sorted based on their light scatter (forward and side scatter) properties, staining using CD14, CD11b or CD11c, or by negative selection by forming rosettes between erythrocytes and unwanted PBMC that can be removed by gradient centrifugation using Ficoll or Lymphoprep, as known in the art. In some embodiments, the monocytes are CD1a⁻/CD14⁺ monocytes. For example, but not limited to, CD1a⁻/CD14⁺ monocytes can be isolated by either positive selection using, for example, anti-CD14-Ig coupled magnetic microbeads (MACS CD14 MicroBeads, human (130-050-201); Miltenyi Biotec, Germany) or negative depletion (MACS Monocyte Isolation Kit II, human (130-91-153); Miltenyi Biotec, Germany).

The DCs used herein can be differentiated from monocytes using various combinations of growth factors known in the art. Suitable growth factors for differentiating monocytes into dendritic cells include, but are not limited to, for example, recombinant human granulocyte macrophage-colony stimulating factor (GM-CSF), recombinant human IL-4, and combinations thereof, among others. DCs may also be derived from CD34⁺ hematopoietic stem or progenitor cells (HSPCs), or from pluripotent stem cells, for example, embryonic stem cells or induced pluripotent stem cells by methods known in the art. Precursor cells may also be isolated from bone marrow and differentiated into DCs as known in the art. Suitable methods of differentiating DCs from CD34⁺ HSPCs are known in the art and include Bedke N, Swindle E J, Molnar C, Holt P G, Strickland D H, Roberts G C, Morris R, Holgate S T, Davies D E, Blume C. A method for the generation of large numbers of dendritic cells from CD34⁺ hematopoietic stem cells from cord blood. J Immunol Methods. 2020 February; 477:112703. doi: 10.1016/j.jim.2019.112703. Epub 2019 Nov. 9. PMID: 31711888; PMCID: PMC6983936, and Curti A, Fogli M, Ratta M, Tura S, Lemoli R M. Stem cell factor and FLT3-ligand are strictly required to sustain the long-term expansion of primitive CD34⁺DR⁻ dendritic cell precursors. J Immunol. 2001 Jan. 15; 166(2):848-54. doi: 10.4049/jimmunol.166.2.848. PMID: 11145659, the contents of which are incorporated by reference in its entirety with regard to methods of differentiating DCs. Suitable methods of differentiating DCs from induced pluripotent stem cells are known in the art and include, for example, those as described in Choi K D, Vodyanik M, Slukvin I I. Hematopoietic differentiation. 2012 Jun. 10. In: StemBook [Internet]. Cambridge (Mass.): Harvard Stem Cell Institute; 2008-. PMID: 23658971, Choi K D, Vodyanik M, Slukvin I I. Hematopoietic differentiation and production of mature myeloid cells from human pluripotent stem cells. Nat Protoc. 2011 March; 6(3):296-313. doi: 10.1038/nprot.2010.184. Epub 2011 Feb. 17. PMID: 21372811; PMCID: PMC3066067, Vodyanik M A, Slukvin I I. Directed differentiation of human embryonic stem cells to dendritic cells. Methods Mol Biol. 2007; 407:275-93. doi: 10.1007/978-1-59745-536-7_19. PMID: 18453262, and U.S. Pat. Nos. 7,811,821, 8,846,395 and 8,435,785, the contents of which are incorporated by reference in their entireties. Induced pluripotent stem cells may be derived from a subject, in some embodiments, the subject may be a donor subject. In some embodiments, the iPSC-derived DCs are further genetically modified to express limited HLA allotypes, and chosen to match the HLA of the subject to be treated. In other embodiments, the subject may have a condition associated with an antigen that is to be treated using the multicell conjugate described herein, and thus the DCs are autologous to the subject to be treated.

The isolated DCs are incubated or contacted in culture with an antigen prior to, during or after co-culturing with iNKT cells and the formation of the multicell conjugate. In one embodiment, the DC are incubated with the antigen prior to conjugation with the iNKT. In another embodiment, the DC are incubated concurrently with the antigen and the iNKT cells to form the conjugate. In a further embodiment, the iNKT+DC conjugate is formed, and subsequently exposed to the antigen prior to administration to a subject. DCs are loaded with a source of antigens to allow for processing and presentation of the antigen or fragment thereof by the DC on its surface. It is this presentation of antigen on the DC surface that can, when in contact with T cells, activate an antigen-specific population of T cells in the subject. The terms antigen “loading” refers to the process by which DCs, when contacted with an antigen, take up, process and express or present the antigen or a fragment thereof on the DC surface (through binding to the major histocompatibility complex (MHC) class I or II molecules). The antigens may be added to the DC culture medium in any suitable form that can be taken up and processed by a DC. For example, the antigen may be in the form of whole or partial inactivated cells that express the antigen (e.g., tumor cells), whole proteins, the whole proteins either derived directly from antigen-expressing cells or produced recombinantly, synthetic peptides or molecules, among others. In some embodiments, the antigen may be delivered using a DNA/RNA construct to allow a peptide or protein to be synthesized and expressed by the DC (e.g., engineered DCs that contain an exogenous polynucleotide that is capable of being translated into an antigen protein or peptide within the DC). Suitable methods of introducing exogenous polynucleotides are known in the art, including, but not limited to, viral transduction, DNA/RNA transfection, and may be in the form of a plasmid, vector, or other polynucleotide sequence. The DC in turn loads the antigen or fragment thereof into MHC molecules that are transported to and maintained for some time on its surface, resulting in antigen-presenting DC (which also can be referred to as an “antigen loaded,” DC). Methods of loading DC in culture are known in the art and can be performed by one skilled in the art. Suitable antigens for use are described below in the methods of use section depending on the condition to be treated in the subject.

iNKT Cells

iNKT cells can be isolated from a subject, for example, from peripheral blood. iNKT cells are rare in blood, typically comprising just about 0.01-1% of peripheral blood mononuclear cells (PBMCs). However, iNKT cells used in the present invention can be obtained from a sample of blood or other tissue, for example, a blood sample from a healthy donor, and expanded ex vivo in tissue culture, as described below. In alternative embodiments, the iNKT cells can be differentiated from pluripotent stem cells.

Human iNKT cells and certain lymphocytes that are not T cells (e.g. natural killer (NK) cells) share some cell surface molecules, but iNKT cells are a distinct population of T cells that express a semi-invariant αβ T-cell receptor (TCR) composed of a TCRa chain (TRAV10-TRAJ18, also known as Vα24-Jα18) preferentially coupled with a TCRβ chain that contains TRBV25 (also known as Vβ11). While conventional T cells recognize antigens presented by major histocompatibility complex (MHC) molecules, iNKT cells recognize lipid or glycolipid antigens presented by CD1d, a non-polymorphic MHC class I-like molecule. Characteristically, iNKT cells express one or more markers commonly found on T lymphocytes (e.g. CD2, CD3, CD5), but do not express B cell markers such as CD19. Known methods suitable for detecting surface expression of the markers used to identify iNKT cells include but are not limited to flow cytometry. iNKT cells are also characterized in that they recognize, and are potently activated by, α-galactosylceramide (α-GalCer) a glycolipid, and related compounds containing an unusual structure of the sugar moiety.

Methods of obtaining iNKT cells from a subject are known in the art and include, for example, separating iNKT cells from PBMCs isolated from a subject (via a blood sample). Suitable methods of isolating iNKT cells are known in the art, for example, by labeling the cells with an antibody that specifically recognizes the semi-invariant type of TCR used by iNKT cells or with a recombinant CD1d molecule loaded with α-GalCer or a related lipid, and sorting (e.g., via magnetic cell sorting or flow cytometric cell sorting) the iNKT cells from other cells within the PBMCs. For example, as described in the Examples below, a PBMC sample obtained from a subject can be labelled with commercially-obtained fluorescently labeled antibodies against CD3, CD4, CD19, lipid-loaded recombinant CD1d reagent (e.g., human CD1d PBS-57 PE, referred to as “CD1d-tetramer”), or combinations thereof, and sorted (e.g., flow cytometrically sorted) for CD4⁺CD3⁺CD1d-tetramer⁺ iNKT cells (e.g., cells staining positive for CD4, CD3, and CD1d tetramer). Once isolated, the CD4⁺CD3⁺CD1d-tetramer⁺ iNKT cell population is a substantially pure (e.g., greater than 95% pure, preferably greater than 98% pure, alternatively at least 99% pure) population of CD4⁺ iNKT cells.

Once isolated, the iNKT cells can be expanded ex vivo. Methods of expanding iNKT cells ex vivo are understood by one skilled in the art. For example, after iNKT cells are isolated from PBMCs from a subject, the iNKT cells are cultured in the presence of one or more stimulating agents capable of activating the iNKT cell to proliferate. Suitable stimulating agents that are capable of allowing iNKT cells are known in the art. For example, in one embodiment, non-proliferating feeder cells (including cells that are either autologous or allogeneic to the iNKT cells) and agents that deliver a signal to the T cell receptor in media containing IL-2 is used as the one or more stimulating agents expand the iNKT cells. Other methods are known in the art, including, for example, beads labeled with lipid-loaded recombinant CD1d molecules or specific stimulating antibodies (e.g., East J E, Sun W, Webb T J. Artificial antigen presenting cell (aAPC) mediated activation and expansion of natural killer T cells. J Vis Exp. 2012 Dec. 29; (70):4333. doi: 10.3791/4333. PM1D: 23299308, and Exley M A, Hou R, Shaulov A, Tonti F. Dellabona P, Casorati G, Akbari O, Akman H O, Greenfield E A, Gumperz J E, Boyson J E, Balk S P, Wilson S B. Selective activation, expansion, and monitoring of human iNKT cells with a monoclonal antibody specific for the TCR alpha-chain CDR3 loop. Eur J Immunol. 2008 June; 38(6):1756-66. doi: 10.1002/eji.200737389, PMID: 18493987, the contents of which are incorporated by reference in their entireties. Another embodiment contemplated is that non-proliferating cells that have been genetically engineered to express CD1d and co-stimulatory ligands, (see, e.g., Exley M A, Hou R, Shaulov A, Tonti E, Dellabona P, Casorati G, Akbari O, Akman H O, Greenfield E A, Gumperz Boyson J E, Balk S P, Wilson S B. Selective activation, expansion, and monitoring of human iNKT cells with a monoclonal antibody specific for the TCR alpha-chain CDR3 loop. Eur. J Immunol. 2008 June; 38(6): 1756-66, doi: 10.1002/eji.200737389. PMCID: 18493987; PMCID: PMC2864538, incorporated by reference in its entirety, etc.), among others. “Non-proliferating feeder cells” and methods of making them are known in the art. In one example, the non-proliferating feeder cells can contain PBMCs isolated from a subject, for example, a healthy adult donor. In some embodiments, the feeder cells are from the iNKT cell donor. The feeder cells are treated by methods known in the art to stop proliferation of the unwanted cells (e.g., feeder cells) prior to culturing with the iNKT cells (e.g., irradiated prior to co-culture). The feeder cells provide culture components (e.g., cytokines and other growth factors, cell surface ligands) to allow for iNKT cell expansion in in vitro culture. Suitable methods of preparing a feeder cell layer are known in the art, for example, PBMCs are irradiated with about 70 Gy ionizing radiation using a gamma particle irradiator, such as an X-ray irradiator or cesium irradiator and resuspended in media containing IL-2 and activating compounds (e.g. phytohemagglutinin (PHA), or specific antibodies) prior to culturing with the iNKT cells. In other embodiments, conditioned medium from feeder cells, or recombinant cytokines (e.g. IL-7, IL-12, IL-15, IL-18) can be used as the one or more stimulating agents.

iNKT cells and at least one stimulating agent/factor (e.g. feeder cells, beads, antibodies, PHA, etc. and IL-2) are provided in a tissue culture plate and cultured for a time sufficient for the iNKT cells to proliferate. Suitable concentrations of IL-2 for proliferating iNKT cells in culture are known in the art, for example, from about 5U/ml to about 1000 U/ml, preferably about 25 to about 400 U/ml, preferably about 200 U/ml.

Preferably, the iNKT cells are cultured and the conjugates are formed in chemically defined medium, preferably in xenogen-free medium. In some embodiments, the medium is serum free. In a further embodiment, the medium is serum-free and xenogen-free. The term chemically defined refers to medium in which all the components within the medium are known. Suitable xenogen-free medium are known in the art and include, but are not limited to, for example, X-VIVO-10 (Lonza), CTS OpTmizer T cell Expansion SFM or AIM C Medium (Gibco).

One or more stimulating agents are added to the iNKT/feeder culture. Suitable stimulating agents include, but are not limited to, for example, phytohemagglutinin (PHA), anti-CD3 mAb, iNKT-TCR specific antibody (e.g. 6B11), recombinant CD1d molecules loaded with α-GalCer (or other related compounds such as PBS 57), engineered cell lines that express CD1d and other key activating molecules, among others. Suitable stimulating agents are known in the art and would be contemplated for use in the present invention. In one embodiment, one or both of the following stimulating agents is added to the iNKT/feeder culture: phytohemagglutinin at a final concentration of 1-5 μg/ml, or 10-30 ng/ml anti-CD3 mAb (e.g., clone OKT3 or SPVT3b). Cells are cultured for a time sufficient to allow for proliferation of the iNKT cells. Cells may be cultured for at least 2 weeks, alternatively at least 4 weeks, alternatively at least 6 weeks, alternatively at least 8 weeks. iNKT cells can be cultured as long as the iNKT cells appear to be increasing in number while cultured. Further, once iNKT cells appear to slow in proliferation, the iNKT cells can be re-stimulated by adding the one or more stimulating agents again to increase proliferation. Many such re-stimulations can be performed sequentially over time, such that a culture of iNKT cells can be expanded over a period of a year or more. The iNKT cells are split into additional cultures when the cells reach confluence on the tissue culture dish. iNKT cells can be split when they achieve a density of more than 0.5×10⁶ per cm² (in a volume of 1 ml per cm²), and should be split when they are at 2×10⁶ per cm² or more. If necessary, the iNKT cells can be further purified from the culture by flow cytometric or magnetic sorting to remove contaminating cells and obtain a substantially pure population of iNKT cells, e.g., greater than 95% INKT cells, preferably greater than 98%, most preferably at least 99% iNKT cells (as demonstrated by staining for one or more iNKT cell markers, e.g., CD4, CD3, and/or CD1d-tetramer (i.e., CD4⁺ iNKT cells)). In some embodiments, the methods comprise (a) sorting CD1d-tetramer⁺CD4⁺CD3⁺ iNKT cells from peripheral blood mononuclear cells obtained from a blood sample of the subject, and (b) expanding the sorted CD1d-tetramer⁺CD4⁺CD3⁺ iNKT cells in culture.

In another embodiment, the iNKT cell can be generated from expansions of a single sorted iNKT cell (i.e., clonal culture). Methods of sorting iNKT cells are known in the art and described herein (e.g., FACS, magnetic sorting, etc.). In some embodiments, multiple iNKT clonal cultures (i.e. populations derived from a single cell) may be mixed together prior to use in forming the conjugates.

In some embodiments, the iNKT cells are frozen in aliquots at a concentration of about 1-20×10⁶ cells/ml in freezing medium and stored at −80° C. until thawed for use in making multicell conjugates. Suitable freezing medium are known in the art and are commercially available, including, for example, CryoStor CS10 freezing medium (StemCell Technologies), Cryoprotective Freezing Medium (Lonza), Recovery Freezing Medium (Gibco), or clinical grade CTS Synth-a-Freeze (Gibco), among others.

It is envisioned that iNKT cells can be cultured, expanded and then frozen until a later time at which the multicell conjugate is made.

### Multicell Conjugate and Compositions

The multicell conjugates are prepared by combining human iNKT cells (isolated from blood of healthy subjects and expanded in tissue culture) with human DCs (for example, DCs generated by differentiating freshly isolated monocytes into DCs in tissue culture). In some embodiments, the iNKT cells and DC are cultured in conditions such that at least 20% of the cells tightly adhere into conjugates containing 2 or 3 cells. In other embodiments, the culture can be selected or sorted for doublets or doublets and triplets comprising iNKT cells and DCs to obtain a pure conjugate preparation. The DCs as described above may be loaded with one or more antigens prior, during or after conjugation. In a preferred embodiment, the DCs are incubated with iNKT cells and antigen concurrently. For example, in one embodiment, the method comprises (a) obtaining an iNKT cell from a subject; (b) expanding the iNKT cell in culture: (c) freezing the expanded iNKT cells for storage; (d) collecting monocytes from a second subject; (e) culturing the monocytes in vitro using conditions to induce differentiation into DCs; (f) thawing the frozen iNKT cells; and (g) co-culturing the thawed iNKT cells with DCs and antigens at a ratio sufficient to form multicell iNKT+DC conjugates. In another embodiment, the method comprises (a) obtaining an iNKT cell from a subject; (b) expanding the iNKT cell in culture: (c) freezing the expanded iNKT cells for storage; (d) collecting monocytes from a subject; (e) culturing the monocytes in vitro using conditions to induce differentiation into DCs; (f) contacting the DCs with antigen; (g) thawing the frozen iNKT cells; and (h) co-culturing the thawed iNKT cells with antigen-contacted DCs at a ratio sufficient to form multicell iNKT+DC conjugates.

In another embodiment, the human DCs (for example, DCs generated by differentiating freshly isolated monocytes into DCs in tissue culture) are exposed to medium containing human serum albumin (HSA) or cytokines prior to incubation with the iNKT cells. This pre-treatment step of the DCs is able to increase the amount of multicell conjugates generated during co-culture with iNKT cells. For example, but not limited to, the methods may use highly purified or recombinant human serum albumin (HSA) containing bound lipids including lysophospholipids, or may use cytokines such as Tumor Necrosis Factor-α (TNF-α), Interleukin-1β (IL-1β), or Interferon-γ (IFN-γ). The DCs are cultured in the presence of HSA or cytokines for a sufficient time prior to iNKT conjugation such that the pre-incubation step increases the amount of multicell conjugates formed. In one method, the DCs are cultured for 1-8 hours in a serum-free medium (for example, X-VIVO-10 (Lonza), CTS OpTmizer T cell Expansion SFM or AIM C Medium (Gibco)), then HSA is added to the medium and the DCs are cultured for an additional 1-8 hours before being combined with iNKT cells. In another method, cytokines such as TNF-α, IL-1β, and/or IFN-γ are added to the DC culture for 1-8 hours before the DCs are combined with iNKT cells. For example, after incubation with iNKT cells, at least 20% of the DCs in the culture are within multicell conjugates by the methods described, alternatively at least 40%, alternatively at least 60% or more. This method produces sufficient multicell conjugates that the cell preparations can be used without a further purification or isolation step for the conjugates. Again, the DC as described above are loaded with an antigen prior, during or after conjugation. In a preferred embodiment, the DCs are incubated with the antigen concurrently with the iNKT cells. For example, in one embodiment, the method comprises (a) obtaining an iNKT cell from a subject; (b) expanding the iNKT cell in culture: (c) freezing the expanded iNKT cells for storage; (d) collecting monocytes from a second subject; (e) culturing the monocytes in vitro using conditions to induce differentiation into DCs; (f) thawing the frozen iNKT cells; (g) treating the DCs with serum-free medium and HSA and/or added cytokines; and (h) co-culturing the thawed iNKT cells with the pre-treated DCs and antigens at a ratio sufficient to form multicell conjugates of iNKT+DC cells. This method would preferably not require an additional isolation or separation step before use of the multicell conjugates. In some embodiments, the HSA is left in the medium during iNKT and DC conjugation. In other embodiment, the HSA is not provided in the medium during conjugation.

In some embodiments, the multicell conjugates are formed under conditions in which the iNKT and DC cells in culture form conjugates (e.g., more that 20% of the culture is conjugates, alternatively more than 40% of cells in culture are conjugates, alternatively at 50% of cells in culture are conjugates). Not to be bound by any theory, but, in some embodiments, the conjugates of the present invention may be used in the methods of treatment as described more herein without the need for isolation for the unconjugated cells in the culture.

In further embodiments, the methods described above may further contain a step of selecting or sorting the multicell conjugate from the culture. In one embodiment, the conjugates may be selected by their light scatter in flow cytometry. Other methods of sorting the multicell conjugate are known in the art. For example, suitable methods of sorting the multicell conjugate from the culture in which they are formed include, but are not limited to flow cytometric cell sorting using for example, iNKT and DC expression markers. Additionally, a preferred method would be to avoid using antibodies (and to use NKT cells and DCs that are pre-labeled with different fluorophores. In another embodiment, multicell conjugate would be separated from multi-cellular aggregates that are above a certain size (e.g. containing 4 or more cells) and also exclude material that is equal to or smaller than the size of an intact dendritic cell.

The iNKT+DC conjugates activate antigen-specific human T cells in a contact-dependent manner. This in turn allows for the ability to activate an antigen-specific immune response, specifically an antigen-specific T cell response in a subject in need thereof. In the Examples, the inventors demonstrate that administering the iNKT+DC conjugates in a humanized mouse model of Epstein-Barr virus (EBV) induced human B cell lymphoma results in clearance of tumor masses and exposure to INKT+DC conjugates promotes target cell killing by T cells in vitro. Thus, iNKT+DC conjugates can be loaded with an appropriate source of antigens (e.g., tumor cell antigens or pathogenic (e.g., viral, bacterial, fungal antigens) and administered as a cellular immunotherapy to activate a specific population of T cells within a subject to help treat and ameliorate an antigen associated condition.

In some embodiments, the multicell conjugate described herein secretes the cytokine IL-12p70 into the culture medium along with other factors. In another embodiment, the multicell conjugates express the cytokine IFN-γ into the culture medium. The DCs and iNKT cells of the conjugate may each express certain markers on their surface after conjugation, and in some embodiments may upregulate or downregulate certain markers or stimulatory factors as compared to their unconjugated counterparts. For example, the cells of the multicell conjugate can express MEW class I and class II, CD40, CD70, CD80, CD83, CD86, CD134L (OX40L), CD137L (4-1BBL), CD215 (IL-15Rα) or a combination thereof on its surface. In some embodiments, the conjugates exhibited elevated levels of cell surface expression of co-stimulatory ligands CD70, CD80, CD86, CD134L, CD137L, CD215 or combinations thereof and lower expression of the inhibitory ligand PD-L1 as compared to unconjugated DCs. For example, in one embodiment, the iNKT cells of the conjugate express CD70 and the DCs of the conjugate express one or more of the markers selected from CD80, CD86, CD134L, CD137L, CD215, or combinations thereof. Levels of cell surface expression can be determined by methods known in the art, for example, by flow cytometric analysis, immunofluorescence microscopy, and other suitable methods performed by one skilled in the art.

The multicell conjugate and compositions comprising the same can be used to elicit an immune response in a subject. In some embodiments, a CD4⁺ T cell response is elicited. In other embodiments, a CD8⁺ T cell response is elicited. In further embodiments, the multicell conjugate and compositions thereof are used to increase expression of one or more immune activating cytokines within a subject, for example, IL-12p70, INF-γ, etc. The multicell conjugate and compositions thereof can also be used to modify the expression of pro-inflammatory cytokines. Compositions, including pharmaceutical compositions, include the multicell conjugate preparations of the invention are described herein. In addition to the multicell conjugate, such compositions can include a pharmaceutically acceptable carrier. The term “pharmaceutically acceptable” can refer to compositions approved by a regulatory agency (e.g., a federal or state government agency) for administration to a subject. The term “carrier” can refer to a diluent, excipient, or vehicle with which the pharmaceutical composition can be administered. Pharmaceutically acceptable carriers are known in the art and include, but are not limited to, for example, diluents, preservatives, solubilizers, emulsifiers, liposomes, nanoparticles among others. Suitably, the pharmaceutically acceptable carrier maintains the viability of the multicell conjugate for storage and delivery to the subject. Additionally, such pharmaceutically acceptable carriers may be solutions, suspensions, and emulsions in aqueous or non-aqueous solvents. Examples of nonaqueous solvents are propylene glycol, polyethylene glycol, vegetable oils such as olive oil, and injectable organic esters such as ethyl oleate. Suitable aqueous solvent carriers include isotonic solutions, alcoholic/aqueous solutions, emulsions or suspensions, including saline and buffered media. Water alone is not contemplated as a suitable physiologically acceptable carrier. In some embodiments, additional components physiologically acceptable for administration to a subject may be added to preserve the viability of the cells and to maintain the conjugate of the present invention. The compositions may contain additional pharmaceutically acceptable substances as required to approximate physiological conditions such as a pH adjusting and buffering agent, toxicity adjusting agents, such as, sodium acetate, sodium chloride, potassium chloride, calcium chloride, sodium lactate, and the like.

The compositions can be sterilized by conventional, well-known sterilization techniques that maintain the viability of the multicell conjugate. Examples of suitable pharmaceutical carriers are described in “Remington's Pharmaceutical Sciences” by E. W. Martin. The formulation should be selected according to the mode of administration. Buffers can include, but are not limited to, phosphate, citrate, and other organic acids; antioxidants including ascorbic acid; low molecular weight (less than about 10 residues) polypeptide; proteins, such as serum albumin, gelatin, or immunoglobulins; hydrophilic polymers such as polyvinylpyrrolidone; amino acids such as glycine, glutamine, asparagine, arginine or lysine; monosaccharides, disaccharides, and other carbohydrates including glucose, mannose, or dextrins; chelating agents such as EDTA; sugar alcohols such as mannitol or sorbitol; salt-forming counterions such as sodium; and/or nonionic surfactants such as TWEEN™ brand surfactant, polyethylene glycol (PEG), and PLURONICS™ surfactant. Pharmaceutically acceptable carriers can include but are not limited to 0.01 to 0.1 M, preferably 0.05M, phosphate buffer or 0.9% saline. Other suitable pharmaceutically acceptable carriers are also contemplated.

In some embodiments, the composition includes the multicell conjugate and one or more checkpoint inhibitors. Suitable checkpoint inhibitors include, but are not limited to, for example, agents capable of blockade of T cell immune checkpoint receptors, including but not limited to PD-1, PD-L1, TIM-3, LAG-3, CTLA-4, and CSF-1R and combinations of such checkpoint inhibitors. The agents that assert immune checkpoint blockade may be small chemical entities or polymers, antibodies, antibody fragments, single chain antibodies or other antibody constructs, including but not limited to bispecific antibodies and diabodies. Immune checkpoint inhibitors that may be used according to the invention include, but are not limited to, for example, anti-PD-1 antibody, anti-PD-L1 antibody, anti-CTLA4 antibody, anti-LAG-3 antibody, and/or anti-TIM-3 antibody. Approved checkpoint inhibitors in the U.S. include atezolizumab, ipilimumab, pembrolizumab, and nivolumab. Others in Phase 3 clinical trials include tislelizumab. The inhibitor need not be an antibody, but can be a small molecule or other polymer. If the inhibitor is an antibody, it can be a polyclonal, monoclonal, fragment, single chain, or other antibody variant construct. Inhibitors may target any immune checkpoint known in the art, including but not limited to, CTLA-4, PDL1, PDL2, PD1, B7-H3, B7-H4, BTLA, HVEM, TIM3, GALS, LAG3, VISTA, KIR, 2B4, CD160, CGEN-15049, CHK1, CHK2, A2aR, and the B-7 family of ligands. Combinations of inhibitors for a single target immune checkpoint or different inhibitors for different immune checkpoints may be used. Preferably, the checkpoint inhibitor is a PD-1 or a PDL-1 or CTLA-4 inhibitor checkpoint inhibitor. Checkpoint inhibitors are commercially available and known in the art. For example, tremelimumab, an anti-CTL4 antibody is available from MedImmune (AstraZeneca) and described in U.S. Pat. No. 6,682,736 and EP Patent No. 1141028; atezolizumab is an anti-PD-L1 available from Genentech, Inc. (Roche) and described in U.S. Pat. No. 8,217,149; ipilimumab, an anti-CTLA-4 available from Bristol-Myers Squibb Co, described in U.S. Pat. Nos. 7,605,238, 6,984,720, 5,811,097, and EP Patent No. EP1212422, among others; pembrolizumab, and anti-PD-1 antibody, available from Merck and Co and described in U.S. Pat. Nos. 8,952,136, 83,545,509, 8,900,587 and EP2170959; nivolumab, an anti-PD-1 antibody, available from Bristol-Myers Squibb Co and described in U.S. Pat. Nos. 7,595,048, 8,728,474, 9,073,994, 9,067,999, 8,008,449 and 8,779,105; tislelizumab available from BeiGene and described in U.S. Pat. No. 8,735,553; among others.

In some embodiments, this composition is for use in treating cancer, as described more below. The composition may comprise the multicell conjugate alone, or the multicell conjugate and one or more checkpoint inhibitors for the treatment of cancer as described in more detail below. In some embodiments, the conjugates could be administered in conjunction with other immunotherapies. For example, the iNKT+DC conjugates could be administered in conjunction with CAR-T cell immunotherapy, NK cell based immunotherapy, or with antibody-mediated immunotherapy designed to target cancer cells for elimination. Suitable immunotherapies are known in the art, for example, a target cancer therapy is Rituximab, chimeric monoclonal antibody against CD20, found on the surface of immune system B cells, and used to treat certain types of cancers (e.g., non-Hodgkin lymphoma, chronic lymphocytic leukemia, B-cell malignancies, etc. In another embodiment, the conjugates may be administered in combination with other biological agents (e.g. cytokines such as IL-15 or type I or type III interferons) or approaches (e.g. certain radiation protocols) that are designed to activate immune responses.

### Methods of Treatment

The multicell conjugates described herein (and compositions comprising them) can be used to activate human antigen-specific T cell responses in a subject in need thereof. As the iNKT+DC conjugates have elevated cell surface expression levels of a wide variety of ligands that are known to activate T lymphocytes, this allows for increased T-cell activation against specific antigens to which the multicell conjugate are targeted. The multicell conjugates described herein, as demonstrated in the examples, can activate antigen-specific human T cells in a contact-dependent manner. Administering such multicell conjugates in a mouse tumor model showed clearance of tumor masses and exposure to INKT+DC conjugates promotes target cell killing by T cells in vitro. The iNKT+DC conjugate can be used in either an allogeneic or an autologous manner, for example, allogenic iNKT cells can be expanded and frozen for later conjugation with autogenic DC from a subject in need of treatment, against specific antigens to treat the antigen associated condition. In some embodiments, the multicell conjugates and compositions thereof can be used in methods to activated CD4⁺ T cells within a subject. In another embodiment, the multicell conjugates and compositions thereof can be used to activate CD8⁺ T cells in an antigen-dependent matter. In some embodiments, the CD8⁺ and/or CD4⁺ T cells produced an antitumor effect and are able to inhibit or reduce tumor cell growth and proliferation.

The present disclosure provides methods and kits of treating a condition associated with an antigen comprising producing cultured iNKT cells (e.g., “off the shelf” iNKT cells) and methods of use for conjugating to the allogenic iNKT cells to freshly generated monocyte-derived DCs from a patient in need of treatment. For example, the multicell conjugates would be made from DCs loaded with an appropriate source of antigens (e.g., tumor cell antigens or pathogen antigens) conjugated to the iNKT cells and administered as a multicell conjugate (i.e., cellular immunotherapy) to activate the patient's T cells against the antigen.

Thus, in one embodiment, the present disclosure provides method of treating a subject with a condition associated with an antigen. The method comprises administering an effective amount of the multicell conjugate described herein to treat the condition. The multicell conjugate comprises a DC loaded with the antigen associated with the condition, as described above. By “condition associated with an antigen” used herein we mean a condition or disease in which one or more specific antigens is an associated characteristic, and in which a cellular immune response (particularly a T cell response) against one or more antigen results in a reduction, inhibition or elimination of the disease or condition. By “antigen” we mean a molecule that induces an immune response in the body. The term “antigen” can be used interchangeably with the term “immunogen.” Specifically, antigens suited for use in the present invention are antigens that are capable of eliciting a cell-mediated immune response, particularly a T cell response. In some embodiments, the antigen may be a superantigen, as described more herein.

In some embodiments, the antigen is a modified superantigen or fragment thereof. A superantigen is a microbial protein or synthetic protein that can immunomodulate the immune reaction. Superantigens are usually derived from microorganisms such as bacteria, viruses and mycoplasma. Their effects on immune system are obtained through their binding both to an outer portion of MHC molecules on antigen presenting cells and to parts of T cell antigen receptors (TCRs) that do not directly contact antigen. In some instances, superantigens are synthetic peptides mimicking a protein from a microorganism that modulates immune activation. Suitable superantigens may be derived from Streptococcal pyrogenic exotoxins (SPE), Staphylococcal enterotoxins (SE), and enterotoxogenic E. coli (ETEC) enterotoxin, among others and are well known in the art. Superantigens are well known in the art and include, but are in no way limited to the superantigens described or derived from those found in Superantigens: mechanism of T-cell stimulation and role in immune responses. Herman A, Kappler J W, Marrack P, Pullen A M. Annu Rev Immunol. 1991; 9:745-72. doi: 10.1146/annurev.iy.09.040191.003525. PMID: 1832875; Heterologous Chimeric Construct Comprising a Modified Bacterial Superantigen and a Cruzipain Domain Confers Protection Against Trypanosoma cruzi Infection. Antonoglou M B, Sánchez Alberti A, Redolfi D M, Bivona A E, Fernandez Lynch M J, Noli Truant S, Sarratea M B, Iannantuono López L V, Malchiodi E L, Fernandez M M. Front Immunol. 2020 Jun. 30; 11:1279. doi: 10.3389/fimmu.2020.01279. eCollection 2020. PMID: 32695105], incorporated by reference in their entireties.

As described above, the DCs are contacted or loaded with a source of antigen. The loaded-DC thus when administered via the iNKT+DC conjugate can activate an antigen-specific population of T cells depending on the antigen loaded on the DC within the conjugate.

The antigens used at this step will depend on the disease being treated. Relevant antigens can be added to the DC culture medium in the form of whole inactivated tumor cells, whole proteins (e.g., whole proteins either derived directly from tumor cells or produced recombinantly), synthetic peptides or molecules, among others. The DCs take up and process these antigens, i.e., proteins into peptides that they load intracellularly into the DC's MHC molecules, which are then transported to their cell surface. Once on the DC surface, the antigen-loaded MHC molecules when in contact with T cells, are able to interact with and activate antigen-specific T cell response. An advantage of using whole cells or proteins as an antigen source is that it is not necessary to identify specific peptide sequences that can be loaded via the particular MHC molecules (i.e., MHC class II molecules) of that individual's DCs. Whole proteins will be processed intracellularly within the DCs and appropriate antigenic fragments can then be expressed on the DC surface via the MHC molecules. In other embodiments, peptides known to be able to be presented on MHC molecules can be used. In one embodiment, the antigen contacted or incubated with the DC is taken up and processed for expression on MHC class II molecules on the DC, which in turn are able to activate antigen-specific CD4⁺ T cells.

In another embodiment, the antigen incubated with the DC is taken up and presented by MHC class I molecules on the DC (a process known as “cross-presentation”), which in turn allows activation of antigen-specific CD8⁺ T cells. In another embodiment, synthetic peptides can be incubated with the DCs that comprise known antigenic epitopes that bind to specific MHC class I molecules, in turn allowing for the activation of CD8⁺ T cells. In another embodiment, the peptide or protein antigen may be expressed in the cell by an exogenous polynucleotide sequence (e.g., plasmid, viral vector, etc.), or via DNA or RNA transduction or transfection, which allows synthesis, processing and expression of the antigen in the MHC/HLA molecule on the DC surface.

In some embodiments, the condition is cancer and the antigen is a tumor antigen. Specifically, in some embodiments, the multicell conjugate comprises DC obtained from a subject having cancer. In some embodiments, the multicell conjugate comprises a DC expressing MHC II or MHC I bound to the tumor antigen or a fragment thereof.

In one embodiment, the condition associated with an antigen is a cancer or tumor (e.g., the antigen is a tumor antigen or cancer antigen). A tumor antigen is an antigen produced by tumor cells. These antigens can sometimes be expressed only by tumor cells and never by normal non-tumor cells, and thus are called tumor-specific antigens. In some instances, tumor-specific antigens result from a tumor specific mutation. Some tumor-associated antigens (TAA) are antigens that are presented by tumor cells and normal cells, but found at upregulated amounts on the tumor cells. Both tumor-specific antigens and tumor-associated antigens are contemplated for use in the present invention.

Tumor antigens that are selectively associated with tumors fall into three main categories: i) neo-antigens that derive from individual somatic mutations in a particular patient, and that are rarely shared among patients (i.e., patient-specific tumor antigens); ii) proteins that are characteristically expressed by specific types of tumors (i.e., shared tumor-specific antigens), iii) viral proteins derived from cancer-causing viruses. The present invention is contemplated to use one or more tumor antigen. Patient-specific tumor antigens (neo-antigens) may be most effectively delivered by exposing the DCs to material from the patient's own cancer cells. Cancer cells are obtained via biopsy and killed by irradiation or other methods prior to exposure to the DCs. The DCs are exposed directly to the tumor cells or to a total protein extract generated from the tumor cells. Alternatively, specific neo-antigens may be identified by genomic or mass-spectroscopic analyses, and the corresponding proteins/peptides produced synthetically.

Shared tumor antigens include well-characterized tumor associated antigens that are expressed by multiple types of cancers and shared by many different patients include Cancer Testis Antigens such as NY-ESO-1, and members of the melanoma-associated antigen (MAGE) family (e.g., MAGE-A3). Additionally, tumor-specific antigens have been identified that are expressed in unrelated individuals with tumors of a given type (e.g., breast cancers, hematopoietic malignancies).

Numerous tumor antigens are known in the art, including, but not limited to, for example, (i) cancer-testis antigens such as NY-ESO-1, SSX2, SCP1 as well as RAGE, BAGE, GAGE and MAGE family polypeptides, for example, GAGE-1, GAGE-2, MAGE-1, MAGE-2, MAGE-3, MAGE-4, MAGE-5, MAGE-6, and MAGE-12 (which can be used, for example, to address melanoma, lung, head and neck, NSCLC, breast, gastrointestinal, and bladder tumors), (ii) mutated antigens, for example, p53 (associated with various solid tumors, e.g., colorectal, lung, head and neck cancer), p21/Ras (associated with, e.g., melanoma, pancreatic cancer and colorectal cancer), CDK4 (associated with, e.g., melanoma), MUM1 (associated with, e.g., melanoma), caspase-8 (associated with, e.g., head and neck cancer), CIA 0205 (associated with, e.g., bladder cancer), HLA-A2-R1701, beta catenin (associated with, e.g., melanoma), TCR (associated with, e.g., T-cell non-Hodgkins lymphoma), BCR-abl (associated with, e.g., chronic myelogenous leukemia), triosephosphate isomerase, KIA 0205, CDC-27, and LDLR-FUT, (iii) over-expressed antigens, for example, Galectin 4 (associated with, e.g., colorectal cancer), Galectin 9 (associated with, e.g., Hodgkin's disease), proteinase 3 (associated with, e.g., chronic myelogenous leukemia), WT 1 (associated with, e.g., various leukemias), carbonic anhydrase (associated with, e.g., renal cancer), aldolase A (associated with, e.g., lung cancer), PRAIVIE (associated with, e.g., melanoma), HER-2/neu (associated with, e.g., breast, colon, lung and ovarian cancer), alpha-fetoprotein (associated with, e.g., hepatoma), KSA (associated with, e.g., colorectal cancer), gastrin (associated with, e.g., pancreatic and gastric cancer), telomerase catalytic protein, MUC-1 (associated with, e.g., breast and ovarian cancer), G-250 (associated with, e.g., renal cell carcinoma), p53 (associated with, e.g., breast, colon cancer), and carcinoembryonic antigen (associated with, e.g., breast cancer, lung cancer, and cancers of the gastrointestinal tract such as colorectal cancer), (iv) shared antigens, for example, melanoma-melanocyte differentiation antigens such as MART-1/Melan A, gp100, MC1R, melanocyte-stimulating hormone receptor, tyrosinase, tyrosinase related protein-1/TRP1 and tyrosinase related protein-2/TRP2 (associated with, e.g., melanoma), (v) prostate associated antigens such as PAP, PSA, PSMA, PSH-P1, PSM-P1, PSM-P2, associated with e.g., prostate cancer, (vi) immunoglobulin idiotypes (associated with myeloma and B cell lymphomas, for example), and other tumor antigens, such as polypeptide- and saccharide-containing antigens including (i) glycoproteins such as sialyl Tn and sialyl Lex (associated with, e.g., breast and colorectal cancer) as well as various mucins; glycoproteins may be coupled to a carrier protein (e.g., MUC-1 may be coupled to KLH); (ii) lipopolypeptides (e.g., MUC-1 linked to a lipid moiety); (iii) polysaccharides (e.g., Globo H synthetic hexasaccharide), which may be coupled to a carrier proteins (e.g., to KLH), (iv) gangliosides such as GM2, GM12, GD2, GD3 (associated with, e.g., brain, lung cancer, melanoma), etc. Additional tumor antigens which are known in the art include p15, Hom/Me1-40, H-Ras, E2A-PRL, H4-RET, IGH-IGK, MYL-RAR, Epstein Barr virus antigens, EBNA, human papillomavirus (HPV) antigens, including E6 and E7, hepatitis B and C virus antigens, human T-cell lymphotropic virus antigens, TSP-180, p185erbB2, p180erbB-3, c-met, mn-23H1, TAG-72-4, CA 19-9, CA 72-4, CAM 17.1, NuMa, K-ras, p16, TAGE, PSCA, CT7, 43-9F, 5T4, 791 Tgp72, beta-HCG, BCA225, BTAA, CA 125, CA 15-3 (CA 27.29\BCAA), CA 195, CA 242, CA-50, CAM43, CD68\KP1, CO-029, FGF-5, Ga733 (EpCAM), HTgp-175, M344, MA-50, MG7-Ag, MOV18, NB/70K, NY-CO-1, RCAS1, SDCCAG16, TA-90 (Mac-2 binding protein\cyclophilin C-associated protein), TAAL6, TAG72, TLP, TPS, and the like.

In another embodiment, the subject is a subject having cancer or a tumor, and the method comprises administering a multicell conjugate and a checkpoint inhibitor to the subject having cancer. Suitable checkpoint inhibitors include, but are not limited to, for example, agents capable of blockade of T cell immune checkpoint receptors, including but not limited to PD-1, PD-L1, TIM-3, LAG-3, CTLA-4, and CSF-1R and combinations of such checkpoint inhibitors. In some embodiments, the immune checkpoint inhibitors include anti-PD-1 antibody, anti-PD-L1 antibody, anti-CTLA4 antibody, anti-LAG-3 antibody, and/or anti-TIM-3 antibody. Suitable inhibitors include, for example, tremelimumab, atezolizumab, ipilumumab, pembrolizumab, and nivolumab, tislelizumab, among others. The inhibitor need not be an antibody, but can be a small molecule or other polymer. In a preferred embodiment, the checkpoint inhibitor is a PD-1 inhibitor, PD-L1 inhibitor, CTLA-4 inhibitor, or the like. Suitable PD-1 inhibitors include, but are not limited to, for example, anti-PD-1 antibodies, e.g., pembrolizumab (Keytruda), Nivolumab (Opdivo), and Cemiplimab (Libtayo), among others. Suitable anti-PD-L1 inhibitors, include, but are not limited to, for example, anti-PD-L1 antibodies, including, but not limited to, Atezolizumab (Tecentriq), Avelumab (Bavencio), and Durvalumab (Imfinzi), among others.

By “cancer” or “tumor” we mean any abnormal proliferation or uncontrolled growth of cells, including solid and non-solid tumors. The methods of the present invention can be used to treat any cancer, any metastases thereof, and any chemo-residual growth thereof, including, but not limited to, carcinoma, lymphoma, blastoma, sarcoma, and leukemia. Suitable cancers able to be treated by the multicell conjugate and compositions, methods and kits described herein include, but are not limited to, lymphoma, breast cancer, prostate cancer, colon cancer, squamous cell cancer, small-cell lung cancer, non-small cell lung cancer, ovarian cancer, cervical cancer, gastrointestinal cancer, pancreatic cancer, glioblastoma, liver cancer, bladder cancer, hepatoma, colorectal cancer, uterine cervical cancer, endometrial carcinoma, salivary gland carcinoma, mesothelioma, kidney cancer, vulval cancer, pancreatic cancer, thyroid cancer, hepatic carcinoma, skin cancer, melanoma, brain cancer, neuroblastoma, myeloma, various types of head and neck cancer, acute lymphoblastic leukemia, acute myeloid leukemia, Ewing sarcoma, and peripheral neuroepithelioma. The composition and methods of the present disclosure can also be utilized to treat non-solid tumor cancers such as non-Hodgkin's lymphoma, leukemia and the like.

The term “metastasis,” “metastatic tumor” or “secondary tumor” refers to cancer cells that have spread to a secondary site, e.g., outside of the primary tumor tissue. Secondary sites include, but are not limited to, the lymphatic system, skin, distant organs (e.g., liver, stomach, pancreas, brain, etc.) and the like.

The present disclosure also provides methods of reducing or inhibiting cancer cell growth in a subject having cancer, the method comprising administering an effective amount of the multicell conjugate or composition described herein to reduce or inhibit cancer cell growth, wherein the multicell conjugate comprises a DC loaded with a tumor antigen. Suitable methods of obtaining a tumor antigen-specific multicell conjugate are described herein.

For purposes of the present invention, “treating” or “treatment” describes the management and care of a subject for combating the disease, condition, or disorder. Treating includes the administration of the multicell conjugate or composition described herein to reduce, prevent, ameliorate and/or improve the onset of the symptoms or complications, alleviating the symptoms or complications, or reducing or eliminating the disease, condition, or disorder.

For example, treating cancer in a subject includes the reducing, repressing, delaying or preventing cancer growth, reduction of tumor volume, and/or preventing, repressing, delaying or reducing metastasis of the tumor. Treating cancer in a subject also includes the reduction of the number of tumor cells within the subject. The term “treatment” can be characterized by at least one of the following: (a) reducing, slowing or inhibiting growth of cancer and cancer cells, including slowing or inhibiting the growth of metastatic cancer cells; (b) preventing further growth of tumors; (c) reducing or preventing metastasis of cancer cells within a subject; and (d) reducing or ameliorating at least one symptom of cancer. In some embodiments, the optimum effective amount can be readily determined by one skilled in the art using routine experimentation.

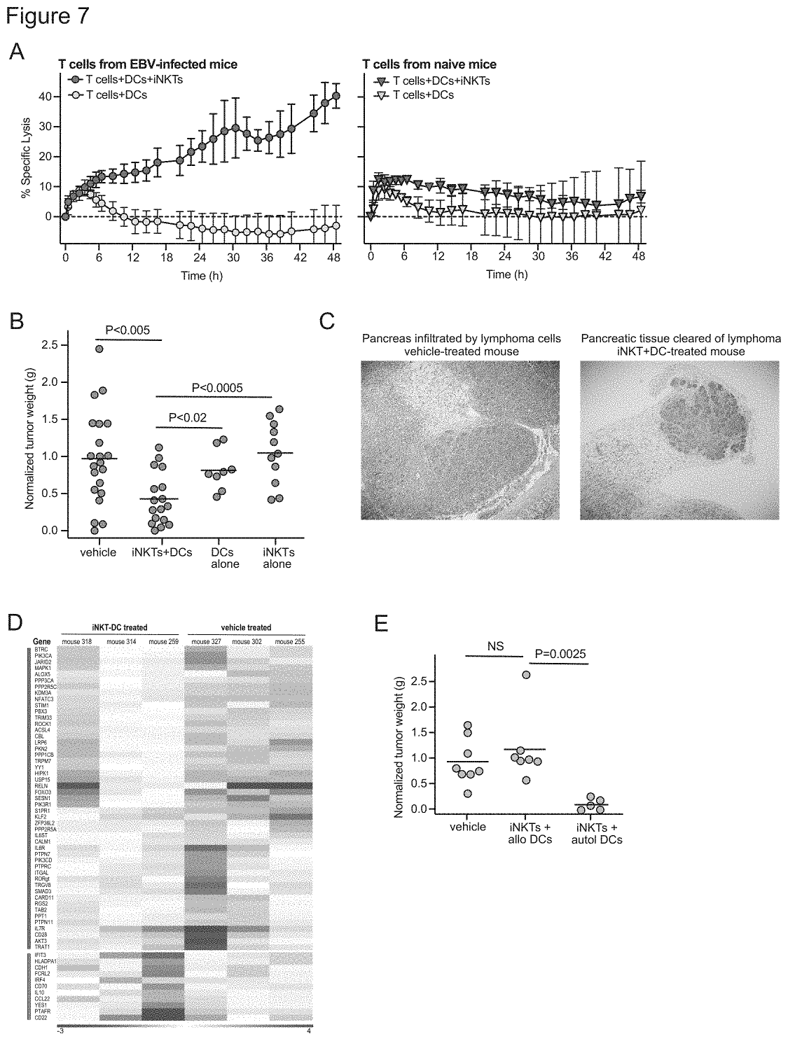

In some embodiments, the methods described herein have an anti-tumor cancer effect that results in a change in the immune genes that are regulated after treatment with the iNKT-DC conjugates. In some embodiments, the disclosure provides a method of modifying an immune response in a subject, the method comprising administering the multicell conjugate or composition thereof to a subject in a sufficient amount to modify an immune response as demonstrated by the upregulation and/or downregulation of the expression of genes that determine immunological functions. An Example is shown in FIG. 7D where modulation of human immune gene expression occurred following administration of iNKT-DC conjugates to mice bearing EBV-induced human B cell lymphomas. As shown in this Example, following iNKT-DC treatment, multiple genes associated with T cell activation were upregulated, including signaling molecules such as kinases (PIK3 isoforms, MAPK1, AKT3) and the calcium channel regulator STIM1, transcription factors (NFAT3C, FOXO3, KLF2), and cell surface receptors that regulate T cell activation and trafficking (CD28, ITGAL, S1PR1). Genes that were downregulated following iNKT-DC treatment included genes associated with EBV-infected B cells including IL-10, FCRL1, and CD22. Thus, in some embodiments, the multicell conjugates and compositions described herein can be used to alter the expression of immune cell genes, the method comprising administering a sufficient amount of the multicell conjugates or compositions described herein.

In another embodiment, the methods use a compound that functions like a modified superantigen to specifically activate a subset of T lymphocytes by forming a bridge between their T cell receptors (TCRs) and MHC molecules expressed on iNKT-DC conjugates. In this embodiment, the DCs are incubated with a synthetic peptide or polypeptide before, during or after conjugation with the iNKT cells. The multicell conjugates bearing the synthetic TCR-MHC bridge can then be administered to a subject, thereby allowing for activation of a subset of the T cells found in the subject.

In another embodiment, the condition associated with an antigen is a pathogen infection. The method comprises administering an effective amount of the multicell conjugate or composition comprising the multicell conjugate in order to treat the pathogen infection. In some embodiments, the DC of the multicell conjugate is contacted with at least one pathogen antigen before formation of the construct and administration to the subject. The term “treating a pathogen infection” includes reducing, inhibiting or preventing the growth of the pathogen and/or reducing or inhibiting one or more symptoms of the pathogen infection.

A pathogen infection is a disease causes by a pathogen. The term “pathogen” refers to an infectious microorganism or agent, for example, a virus, bacterium, protozoan, or fungus. In a preferred embodiment, the pathogen infection is a virus infection. In another embodiment, the infection is a bacterial infection.

Pathogen antigens are antigens associated with the pathogen, for example, viral antigens, bacterial antigens, protozoan antigens and fungal antigens.

In some embodiments, the pathogen infection results in cancer, for example, a number of viruses have been associated with different cancers, two of the best characterized are human papilloma virus (HPV) and Epstein-Barr virus (EBV). HPV is associated with cervical, anal, and head and neck cancers. The most prevalent oncogenic strain of HPV is HPV-16, and the E7 protein of this strain is well characterized as a tumor-associated antigen. EBV is associated with diffuse large B cell lymphoma, post-transplant lymphoproliferative disease, Hodgkin lymphoma, Burkitt lymphoma, nasopharyngeal carcinoma, and gastric cancers. EBV proteins that have been well characterized as tumor antigens recognized by T cells include LMP1 and LMP2A.

Suitable bacterial antigens include, for example, proteins, polysaccharides, lipopolysaccharides, and outer membrane vesicles isolated, purified or derived from bacteria. In addition, bacterial antigens may include bacterial lysates and inactivated bacteria formulations. Bacteria antigens may also be produced by recombinant expression. Bacterial antigens preferably include epitopes that are exposed on the surface of the bacteria during at least one stage of its life cycle. Suitable bacterial antigens include, but are not limited to, for example, antigens derived from one or more of Borrelia burgdorferi, Helicobacter pylori, Mycobacterium tuberculosis, Streptococcus pneumoniae, Neisseria meningitidis, Streptococcus pyogenes (Group A Streptococcus), Moraxella catarrhalis, Bordetella pertussis, Staphylococcus aureus, Clostridium tetani (Tetanus), Corynebacterium diphtheriae (Diphtheria), Haemophilus influenzae type B (Hib), Pseudomonas aeruginosa, Streptococcus agalactiae (Group B Streptococcus), and Escherichia coli, among others.

Suitable viral antigens include, for example, inactivated (or killed) virus, attenuated virus, split virus formulations, purified subunit formulations, viral proteins which may be isolated, purified or derived from a virus, and Virus Like Particles (VLPs). Alternatively, viral antigens may be expressed recombinantly. Viral antigens preferably include epitopes which are exposed on the surface of the virus during at least one stage of its life cycle, and/or are expressed on virus-producing cells. Suitable viral antigens include, but are not limited to, antigens derived from one or more of the following families, orthomyxovirus (influenza), Pneumovirus (RSV), Paramyxovirus (PIV and Mumps), Morbillivirus (measles), Togavirus (Rubella), Enterovirus (polio), Herpesviruses, HBV, Coronavirus (SARS), and Varicella-zoster virus (VZV), Epstein Barr virus (EBV), Papillomavaviruses (e.g., HPV), HIV, among others.

Another pathogen antigen includes fungal antigens derived from pathogenic fungi. Suitable pathogenic fungus include, but are not limited to, for example, Aspergillus fumigatus, Aspergillus flavus, Aspergillus niger, Aspergillus nidulans, Aspergillus terreus, Aspergillus sydowii, Aspergillus flavatus, Aspergillus glaucus, Blastomyces dematitidis, Blastoschizomyces capitatus, Candida albicans, Candida enolase, Candida tropicalis, Candida glabrata, Candida krusei, Candida parapsilosis, Candida stellatoidea, Candida kusei, Candida parakwsei, Candida lusitaniae, Candida pseudotropicalis, Candida guilliermondi, Cladosporium carrionii, Coccidioides immitis, Blastomyces dermatidis, Cryptococcus neoformans, Geotrichum clavatum, Histoplasma capsulatum, Klebsiella pneumoniae, Paracoccidioides brasiliensis, Pneumocystis carinii, Pythiumn insidiosum, Pityrosporum ovale, Sacharomyces cerevisae, Saccharomyces boulardii, Saccharomyces pombe, Scedosporium apiosperum, Sporothrix schenckii, Trichosporon beigelii, Toxoplasma gondii, Penicillium marneffei, Malassezia spp., Fonsecaea spp., Wangiella spp., Sporothrix spp., Basidiobolus spp., Conidiobolus spp., Rhizopus spp, Mucor spp, Absidia spp, Mortierella spp, Cunninghamella spp, Saksenaea spp., Alternaria spp, Curvularia spp, Helminthosporium spp, Fusarium spp, Aspergillus spp, Penicillium spp, Monolinia spp, Rhizoctonia spp, Paecilomyces spp, Pithomyces spp, and Cladosporium spp., Dermatophytres, including: Epidermophyton floccusum, Microsporum audouini, Microsporum canis, Microsporum distortum, Microsporum equinum, Microsporum gypsum, Microsporum nanum, Trichophyton concentricum, Trichophyton equinum, Trichophyton gallinae, Trichophyton gypseum, Trichophyton megnini, Trichophyton mentagrophytes, Trichophyton quinckeanum, Trichophyton rubrum, Trichophyton schoenleini, Trichophyton tonsurans, Trichophyton verrucosum, Trichophyton violaceum, and/or Trichophyton faviforme, among others.

By “administering”, we mean any means for introducing the multicell conjugate or compositions into the body, preferably into the systemic circulation. Examples include but are not limited to oral, buccal, sublingual, pulmonary, transdermal, transmucosal, as well as subcutaneous, intraperitoneal, intravenous, and intramuscular injection.

A preferred method of administering the multicell conjugate or pharmaceutical compositions of the present invention for treatment of cancer is by intravenous administration. Administering also includes introducing the multicell conjugate or compositions locally to the cancer, for example, but not limited to, by topical treatment or injection into the tumor site (e.g., intratumoral injection (solid tumors), administering to bone marrow (for AML, etc), etc.), or systemically to reach the lymph nodes and other immune modulatory sites.

A preferred method of administering the multicell conjugate or pharmaceutical composition of the present invention to treat a pathogenic infection include intravenous administration or local administration to the pathogen infection site. For example, for pneumonia, local administration to the lungs may be contemplated via inhalation or lavage.

The term “effective amount” or “therapeutically effective amount” refers to an amount sufficient to effect beneficial or desirable biological and/or clinical results. For example, such a result can be reducing, inhibiting or preventing the growth of cancer cells (including drug-resistant or therapy-resistant cancer cells), reducing, inhibiting or preventing metastasis of the cancer cells or invasiveness of the cancer cells or metastasis, or reducing, alleviating, inhibiting or preventing at least one symptom of the cancer or metastasis thereof, or any other desired alteration of a biological system. For a pathogen infection, the result can be reducing, inhibiting, or preventing growth and spread of the pathogen, reducing or preventing one or more symptom of the pathogenic infection, among others.

An “effective treatment” refers to treatment producing a beneficial effect, e.g., amelioration of at least one symptom of the disease or condition. A beneficial effect can take the form of an improvement over baseline, i.e., an improvement over a measurement or observation made prior to initiation of therapy according to the method. For cancer, a beneficial effect can also take the form of reducing, inhibiting or preventing further growth of cancer cells, reducing, inhibiting or preventing metastasis of the cancer cells or invasiveness of the cancer cells or metastasis or reducing, alleviating, inhibiting or preventing at least one symptoms of the cancer or metastasis thereof. Such effective treatment may, e.g., reduce patient pain, reduce the size or number of cancer cells, may reduce or prevent metastasis of a cancer cell, or may slow cancer or metastatic cell growth.

By “subject”, we mean mammals, preferably human. Suitably, the subject is a human in need of treatment. However, veterinary uses are also contemplated with the present invention. “Mammals” means any member of the class Mammalia including, but not limited to, humans, non-human primates such as chimpanzees and other apes and monkey species; farm animals such as cattle, horses, sheep, goats, and swine; domestic animals such as rabbits, dogs, and cats; laboratory animals including rodents, such as rats, mice, and guinea pigs; and the like. The term “subject” does not denote a particular age or sex. In a preferred embodiment, the subject is a human. In a preferred embodiment, the subject has a disease or condition associated with an antigen. In one embodiment, the human has a cancer. In another embodiment, the human has a pathogen infection.

### Kits

In further embodiments, the disclosure provides kits for carrying out the methods described herein. In one embodiment, the kit comprises the multicell conjugate described herein and instructions for use. The kit may be used for treating a condition associated with an antigen.

In some embodiments, a kit comprising the frozen iNKT cells and instruction material for forming multicell conjugates are envisioned. In some embodiments, an off the shelf frozen iNKT cell population can be used to form multicell conjugates with DCs derived from a subject in need of treatment using the multicell conjugate. The instruction material may provide instructions on isolating monocytes from a blood sample from a subject in need of treatment and differentiating the monocytes into DCs. Methods of isolating monocytes from PBMCs and from blood are known in the art and described above. Further, the kit may comprise an antigen or instructions for loading the DC cells with the antigen by incubating the DC cells with an antigen before conjugation with the iNKT cells.

In another embodiment, the kit comprises frozen allogenic iNKT cells and instructions for making the multicell conjugate described herein. The kit may further comprise an antigen, and instructions for isolating monocytes from a blood sample. Additional instructions may be provided on how to differentiate the monocytes into DCs. Suitably, the kit may further comprise media and additional components, e.g., stimulating factors, growth factors, antibodies, etc. for the growth, isolation and production of multicell conjugates described herein. In another embodiment, the kit may further comprise instructions on loading the DC with antigen, and/or instructions for selecting and using an antigen for DC loading.

In another embodiment, a kit comprises the multicell conjugate or composition described herein for the treatment of cancer, and instructions for use. The kit can further comprise a checkpoint inhibitor to use in combination with the multicell conjugate or composition comprising the same.

### ADDITIONAL EMBODIMENTS

In one embodiment, the present disclosure provides a stable in vitro-derived multicell conjugate between an invariant natural killer T (iNKT) cell and a dendritic cell (DC). In some aspects, the conjugate is durable in culture for at least 30 minutes. In some aspects, the conjugate being durable in culture for at least 24 hours. In some aspects, the iNKT cell within the conjugate expresses an appropriately rearranged TCR. In further aspects, the iNKT in the conjugate expresses one or more markers selected from the group consisting of CD4, and CD3. In some aspects, the conjugate is maintained in culture for at least 96 hours. In some aspects, the DC expresses major histocompatibility complex molecules I or II (MHC I or MHC II). In some aspects, the multicell conjugate secretes IL-12p70, INF-γ or both into the culture medium. In some aspects, the DC cell expresses one or more of the molecules selected from CD80, CD83, CD86, CD134L (OX40L), CD137L (41BBL), CD215 (IL-15Rα), and combinations thereof on its surface. In some aspects, the DC within the conjugate expresses one or more co-stimulatory molecules for at least 24 hours in vitro. In some aspects, the iNKT cell expresses CD70 on its surface, preferably for at least 24 hours, preferably at least 96 hours after conjugation. In some aspects, the multicell conjugate comprises two or three cells per conjugate. In some aspects, the DCs has loaded with an antigen, preferably wherein the antigen is selected from a tumor antigen, a pathogenic antigen and a superantigen.

In another aspect, the disclosure provides a composition comprising the multicell conjugate and a pharmaceutically acceptable carrier.

In another aspects, the disclosure provides a method of producing a multicell conjugate between an invariant natural killer (iNK) T cell and a dendritic cell (DC), the method comprising: co-culturing an iNKT cell and a DC for a sufficient amount of time to form a stable multicell conjugate. In some aspects, the iNKT cell and DC are co-cultured at an iNKT:DC ratio of 1:1 to about 5:1. In some aspects, the DC is contacted with lipidated human serum albumin or one or more cytokines prior to co-culturing, wherein the contacting increases the ability of the DC to form DC-iNKT conjugates in in vitro culture. In some aspects, the dendritic cell is obtained from a monocyte by a method comprising: (i) culturing a monocyte in culture media comprising GM-CSF and IL-4 for a sufficient time to differentiate the monocyte into a DC. In some aspects, the monocyte is cultured in (i) for at least two days. In some aspects, the method further comprises: (ii) isolating the monocyte from a blood sample of a subject before differentiation, or (iii) isolating the monocyte from a tissue of a subject. In some aspects, step (iii) comprises isolating monocytes from bone marrow. In some aspects, the monocyte is from a donor subject. In some aspects, the DC is generated by: (i) differentiating CD34⁺ hematopoietic progenitor cells in vitro from a bone marrow sample, G-CSF mobilized peripheral blood, or other tissues into DCs; or (ii) differentiating induced pluripotent stem cells (iPSCs) into DCs. In a some aspects, the DC are generated by: a) differentiating from CD34+ hematopoietic progenitor cells or induced pluripotent stem cells into CD1a−/CD14+ monocytes in culture; b) isolating the CD1a−/CD14+ monocytes of step (a); and c) differentiating the isolated monocytes into DC by culturing in medium comprising GM-CSF and IL4 for a sufficient time, wherein DCs are produced that show elevated CD209 and reduced CD14. In some aspects, (a) the iPSCs are allogeneic from the subject in need; (b) the iPSCs are genetically modified to express a limited selection of MEW molecules; (c) the IPSCs express MHC molecules to match the subject to be treated; or (d) combinations of (a)-(c). In some aspects, the method further comprises: (a) contacting the DC in culture with an antigen prior to co-culturing with the iNKT cell; (b) concurrently contacting the DC with an antigen and co-culturing with the iNKT cell; or (c) contacting the iNKT+DC conjugate with the antigen subsequent to conjugate formation. In some aspects, the antigen is selected from a tumor antigen, a pathogenic antigen and a superantigen. In some aspects, the method further comprises obtaining the iNKT cell from a subject. In some aspects, the iNKT cells is differentiated from induced pluripotent stem cells. In some aspects, the method of obtaining the iNKT cell comprises: (a) sorting CD1d tetramer⁺CD4⁺CD3⁺ iNKT cells from (i) peripheral blood mononuclear cells, (ii) bone marrow, or (iii) tissue from a donor containing lymphocytes; and (b) expanding the sorted CD1d tetramer⁺CD4⁺CD3⁺ iNKT cells in culture. In some aspects, the method comprises isolating peripheral blood mononuclear cells from a blood sample of a subject prior to the step (a) sorting step. In another aspect, the method comprises centrifuging a blood sample to obtain the peripheral blood mononuclear cells. In some aspects, step (b) comprises culturing the sorted iNKT cells with one or more stimulating agents in an amount effective to proliferate the iNKT cell in culture prior to conjugation. In some aspects, the DC is allogenic to the iNKT cell. In some aspects, the method comprises (a) obtaining an iNKT cells; (b) expanding the iNKT cell in culture: (c) freezing the expanded iNKT cells for storage; and (d) subsequent thawing of the frozen iNKT cells prior to co-culturing with DC. In some aspects, the iNKT cells are obtained by: (i) isolating from a donor subject; or (ii) differentiating from induced pluripotent stem cells (iPSCs). In further aspects, the method comprises selecting the multicell conjugate from the culture.

In another aspect, the disclosure provides a method of treating a subject with a condition associated with an antigen, the method comprising administering an effective amount of the multicell conjugate or composition described herein to treat the condition. In some aspects, the DC in the multicell conjugate is loaded with an antigen or a fragment thereof before administration to the subject. In some aspects, the DC in the conjugate is obtained from a subject having the condition associated with an antigen. In some aspects, the condition is cancer and wherein the antigen is a tumor antigen. In some apsects, the multicell conjugate comprises DC obtained from a subject having cancer. In some aspects, the multicell conjugate comprises DC expresses MHC I or MHC II loaded with the tumor antigen or a fragment thereof. In some aspects, the method further comprises administering a checkpoint inhibitor to the subject. In some aspects, the condition is a pathogen infection and wherein the multicell conjugate comprises DC loaded with at least one pathogen antigen or fragment thereof. In some aspects, the multicell conjugate is able to traffic in vivo to the immunogenic site within the subject.

In another aspect, the disclosure provides a method of activating T cells in a subject in need thereof, the method comprising administering an effective amount of the multicell conjugate of or the composition described herein to the subject, wherein T cell are activated. In some aspects, the T cells are CD8⁺ T cells.

In another aspect, the disclosure provides a method of activating an immune response against an antigen within a subject, the method comprising: administering an effective amount of the multicell conjugate or the composition described wherein to the subject, wherein an immune response is activated against the antigen in the subject. In some aspects, the antigen is a tumor antigen. In some aspects, the immune response is a CD4⁺ T cell response. In some aspects, the immune response is a CD8⁺ T cell response. In some aspects, the immune response further comprises an increase in one or more cytokines within the subject.

It should be apparent to those skilled in the art that many additional modifications beside those already described are possible without departing from the inventive concepts. In interpreting this disclosure, all terms should be interpreted in the broadest possible manner consistent with the context. Variations of the term “comprising” should be interpreted as referring to elements, components, or steps in a non-exclusive manner, so the referenced elements, components, or steps may be combined with other elements, components, or steps that are not expressly referenced. Embodiments referenced as “comprising” certain elements are also contemplated as “consisting essentially of” and “consisting of” those elements. The term “consisting essentially of” and “consisting of” should be interpreted in line with the MPEP and relevant Federal Circuit interpretation. The transitional phrase “consisting essentially of” limits the scope of a claim to the specified materials or steps “and those that do not materially affect the basic and novel characteristic(s)” of the claimed invention. “Consisting of” is a closed term that excludes any element, step or ingredient not specified in the claim. For example, with regard to sequences “consisting of” refers to the sequence listed in the SEQ ID NO. and does refer to larger sequences that may contain the SEQ ID as a portion thereof.

All publications, patent applications, patents, and other references mentioned herein are incorporated by reference in their entirety. In the case of conflict, the present specification, including definitions, will control.

Other features and advantages of the invention will be apparent from the description of the preferred embodiments thereof, and from the claims. Unless otherwise defined, all technical and scientific terms used herein have the same meaning as commonly understood by one of ordinary skill in the art to which this invention belongs. Although methods and materials similar or equivalent to those described herein can be used in the practice or testing of the present invention, suitable methods and materials are described below. In addition, the materials, methods, and examples are illustrative only and not intended to be limiting.

### EXAMPLES

### Example 1

We have found that in the presence of DCs, invariant natural killer T cells (iNKT cells) can promote the activation of antigen-specific T cells by dendritic cells (DCs) loaded with an antigen. CD4⁺ iNKT cells were sorted from peripheral blood of a healthy adult subject and expanded in vitro to generate a highly pure culture. DCs were derived from monocytes isolated from peripheral blood samples drawn from healthy adult subjects, who were not genetically related to the iNKT cell donor (i.e. allogeneic to the iNKT cells). DCs were co-incubated with a 1:1 ratio of iNKT cells to allow the formation of conjugates, or were cultured alone. Cultures containing iNKT-DC conjugates or DCs alone were co-incubated with antigens, including Epstein-Barr virus (EBV Ag), Toxic Shock Syndrome Toxin superantigen (TSST SAg), or tetanus toxoid antigen (TT Ag), or were mock-treated (No Ag). They were then co-cultured with T cells that were autologous to the DCs, such that the DCs comprised 2% of the total cells in the culture.

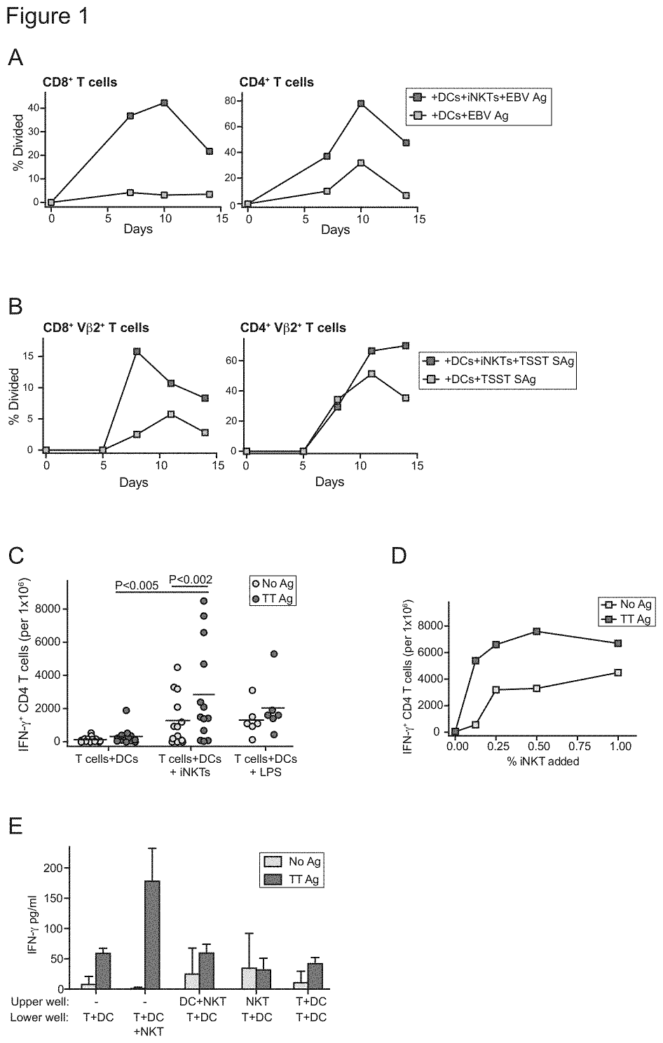

To investigate the ability of antigen-loaded iNKT-DC conjugates to activate T cell proliferation, we labeled the T cells with a fluorescent dye that allows determination of the percent of cells that underwent cell division. As shown in FIGS. 1A and 1B, exposure to iNKT-DC conjugates loaded with EBV antigen resulted in an increased frequency of divided T cells, compared to T cells exposed to EBV antigen loaded DCs alone. Similarly, exposure to iNKT-DC conjugates that were pre-labeled with the TSST superantigen resulted in greater proliferation by the subset of T cells bearing a type of TCR that binds to the TSST superantigen, compared to those that were exposed to TSST-labeled DCs alone. The enhanced proliferation induced by exposure to antigen-bearing iNKT-DC conjugates was observed for both CD8⁺ and CD4⁺ T cells.

To test the ability of antigen-loaded iNKT-DC conjugates to activate cytokine production by T cells, the DCs were pulsed with tetanus toxoid antigen or mock-treated and combined with allogeneic iNKT cells and autologous T cells After 24-48 hours of co-culture, intracellular cytokine staining was performed on the cell mixtures and the samples were analyzed by flow cytometry to determine the frequency of CD4⁺ T cells (excluding iNKT cells) that had begun to produce the cytokine IFN-γ. As shown in FIG. 1C, an increased number of CD4⁺ T cells containing intracellular IFN-γ were observed when iNKT cells were added to the culture containing antigen-treated DCs, demonstrating that iNKT cells promote antigen-dependent activation of CD4⁺ T cells by DCs. The effect of the iNKT cells appeared to be at least as potent as when the DCs were treated with lipopolysaccharide (LPS, a toll-like receptor ligand that is known to provide signals that initiate immune responses), and required that iNKT cells comprise only 0.25% of the culture (FIG. 1D).

To determine whether the stimulatory effect of iNKT cells requires physical contact, DCs were co-cultured with iNKT cells and T cells that either were in the same well or were separated by a transwell membrane. A quantification of IFN-γ secretion revealed that enhanced T cell activation was only observed when the iNKT cells and the DCs were able to contact each other and when the T cells were able to contact both iNKT cells and DCs (FIG. 1E). When the DCs or T cells were exposed only to iNKT-secreted factors enhanced T cell activation was not observed, indicating that the adjuvancy effect of iNKT cells is contact dependent.

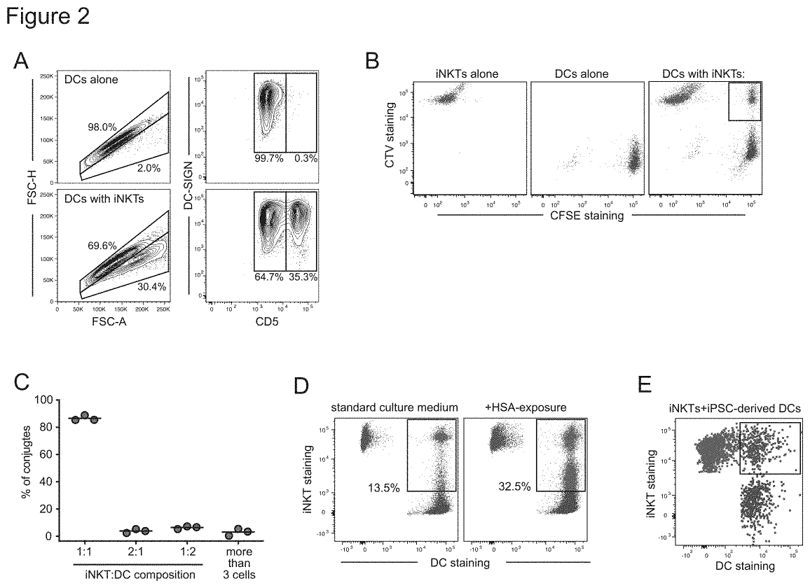

To study the mechanism for this activation, iNKT cells were cultured with DCs. Surprisingly, flow cytometric analysis revealed the presence of a tightly-adhered conjugated population consisting of DCs paired with iNKT cells (FIGS. 2A and 2B). Most conjugates (>80%) contained one iNKT cell and one DC, and less than 10% contained more than three cells total (FIG. 2C). Pre-exposing the DCs to highly purified human serum albumin containing bound lipids including lysophospholipids prior to co-incubation with iNKT cells resulted in a higher frequency of iNKT-DC conjugates (FIG. 2D). Conjugates were also observed between iNKT cells and DCs that were re-differentiated from induced pluripotent stem cells (iPSCs) (FIG. 2E).

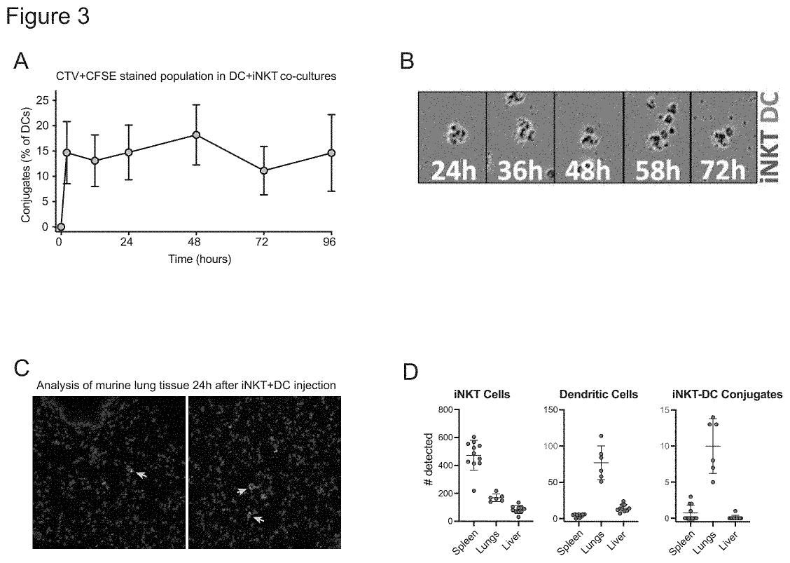

As shown in FIGS. 3A and 3B, conjugates of iNKT cells and DCs remained tightly-adhered for at least 96 hours in culture. Closely associated iNKT cells and DCs (apparent conjugates) were also observed in murine tissues 24 hours after being intravenously injected into immunodeficient mice (FIGS. 3C and 3D).

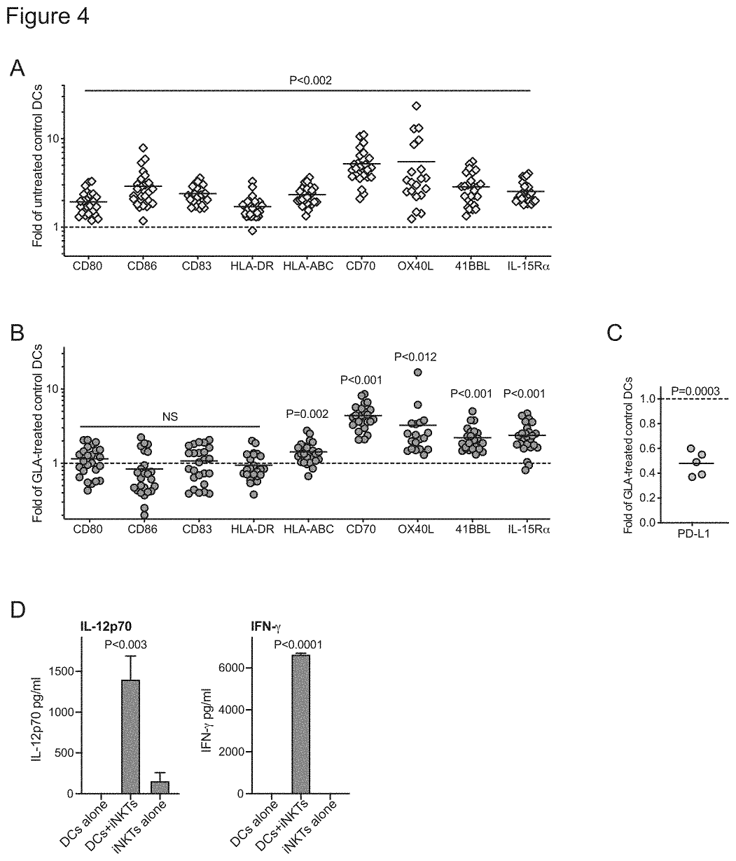

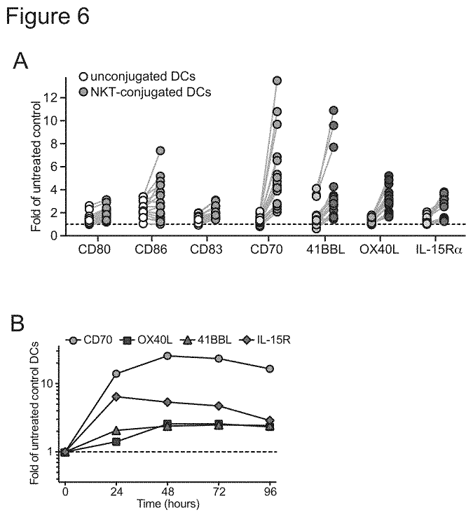

Conjugates of iNKT cells and DCs exhibit elevated cell surface expression levels of a wide variety of ligands that are known to activate T lymphocytes (FIG. 4A). Moreover, compared to DCs that are treated with a synthetic adjuvant (glucopuranosyl lipid adjuvant, or GLA), the iNKT+DC conjugates show higher expression of MHC class I molecules (HLA_ABC), the co-stimulatory ligands CD70, CD134L (OX40L), CD137L (41BBL), CD215 (IL-15Rα) and lower expression of the inhibitory ligand PD-L1 (FIGS. 4B and 4C). This set of co-stimulatory molecules is selectively up-regulated on iNKT cell conjugated DCs and shows little upregulation on DCs that are exposed to iNKT cells but did not form conjugates, and the up-regulated expression on conjugates is maintained for at least 96 hours in vitro (FIG. 6). Secreted IL-12p70 and IFN-γ, cytokines that are known to promote anti-tumor and anti-viral responses by T cells, are elevated in co-cultures of iNKT cells and DCs (FIG. 4D).

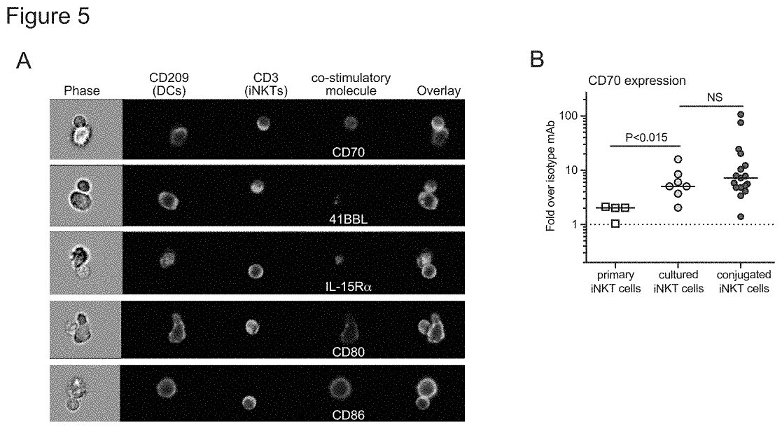

Further analysis using an imaging flow cytometer revealed that both iNKT cells and DCs contribute to the co-stimulatory profile of the conjugates, as the iNKT cells are the component that expresses CD70 and the DCs express other co-stimulatory receptors (FIG. 5A). Additionally, CD70 is upregulated on iNKT cells during culture in vitro, and primary iNKT cells show little CD70 expression directly ex vivo (FIG. 5B).

To test whether the iNKT+DC conjugates could be used to activate T cells to attack autologous tumors, human umbilical cord blood mononuclear cells (including B-lymphocytes, T lymphocytes, monocytes, and DCs) were briefly exposed to Epstein-Barr virus, a human B cell-specific γ-herpes virus that is known to drive the formation of B cell lymphomas, and the cells were injected intraperitoneally into immunodeficient mice. Exposing human T cells harvested from mice bearing EBV-induced lymphomas to iNKT+DC conjugates resulted in enhanced killing of target cells, whereas target cell killing was not enhanced when EBV-naive T cells were exposed to iNKT+DC conjugates (FIG. 7A). To test whether administration of iNKT+DC conjugates promoted clearance of human tumors in vivo, mice bearing EBV-induced B cell lymphomas were injected with iNKT cells alone, DCs alone, iNKTs+DCs, or mock treated. Examination of tumor burden 6 days later revealed that administration of the iNKT+DC mixture leads to reduction of tumor burden, whereas mice that received the same dose of iNKT cells alone or DCs alone showed similar tumor burdens as mock-treated mice (FIG. 7B). Histological analysis of pancreatic tissue from a vehicle-treated mouse revealed extensive lymphocytic infiltration consistent with the presence of a lymphoma tumor, whereas pancreatic tissue from a mouse given iNKT-DC conjugates showed areas that appeared to be cleared of lymphocytic infiltration (FIG. 7C). Spleen tissue was harvested from 3 EBV-lymphoma bearing mice given iNKT-DC conjugates and from 3 EBV-lymphoma bearing mice that were vehicle-treated, and subjected to NanoString Immunoprofiling analysis to assess expression levels of ˜700 human immune-related genes. This analysis revealed 49 genes that were significantly upregulated in mice that were given iNKT-DC conjugates compared to vehicle-treated mice, and 11 genes that were significantly down-regulated in mice that were given iNKT-DC conjugates compared to vehicle-treated mice (FIG. 7D). This analysis revealed that following iNKT-DC treatment, multiple genes associated with T cell activation were upregulated, including signaling molecules such as kinases (PIK3 isoforms, MAPK1, AKT3) and the calcium channel regulator STIM1, transcription factors (NFAT3C, FOXO3, KLF2), and cell surface receptors that regulate T cell activation and trafficking (CD28, ITGAL, S1PR1). Genes that were downregulated following iNKT-DC treatment included genes associated with EBV-infected B cells including IL-10, FCRL1, and CD22. In contrast to the significant anti-tumor effects of administering iNKT cells paired with DCs that were autologous to the T cells and EBV-infected B lymphoma cells, administering iNKT cells and DCs that were allogeneic to the T cells and B cells had no significant anti-tumor effect (FIG. 7E), supporting that the iNKT-DC conjugates activate a specific response by autologous T cells.

### Materials and Methods:

Isolation of iNKT cells: Peripheral blood (15-50 ml) is collected from a healthy adult donor into a heparinized tube (10-30 USP heparin/ml blood). Peripheral blood mononuclear cells (PBMCs) are isolated by density gradient centrifugation (Ficoll-Paque, GE Healthcare), according to the manufacturer's protocol, and suspended in sterile phosphate-buffered saline containing 1% bovine serum albumin (PBS/BSA). PBMCs are labeled for 30 minutes at 4° C. with commercially-obtained fluorescently labeled antibodies against CD3, CD4, CD19, and lipid-loaded CD1d tetramer reagent (e.g., human CD1d PBS-57 PE, from the NIH Tetramer Facility at Emory University). Unbound staining reagents are washed away by pelleting the cells by centrifugation (10 min at 300 g) and resuspending them in sterile PBS/BSA. CD4⁺ iNKT cells (cells staining positive for CD4, CD3, and CD1d tetramer) are flow cytometrically sorted into a solution of RPMI 1640 medium containing 50% bovine calf serum.

Expansion and storage of iNKT cells: PBMCs are isolated from a healthy adult donor to use as “feeder” cells. Notably, it is not necessary for these cells to be from the same person as the subject used to isolate the iNKT cells. The feeder PBMCs are irradiated with 70 Gy ionizing radiation using an X-ray irradiator, and suspended in sterile growth medium prepared according to the following recipe: RPMI 1640 medium diluted with 10% heat-inactivated fetal bovine serum, 5% heat-inactivated bovine calf serum (defined/supplemented), 3% pooled human AB serum, 1% L-glutamine (2 mM final concentration), 1% Penn/Strep (100 IU/ml Penicillin and 100 μg/ml Streptomycin final concentration), 200 U/ml recombinant human IL-2, (final glucose concentration of growth medium should be ˜8.9 mM). Irradiated feeder PBMCs suspended in growth medium are added to sterile polystyrene tissue culture plates at a concentration of 1×10⁶ cells/ml. Sorted iNKT cells are added to a final concentration of 5×10³-5×10⁵ cells/ml. One or both of the following stimulating agents are added to the wells: phytohemagglutinin at a final concentration of 1-5 μg/ml (determined by prior titration experiments using polyclonal T cells from adult PBMCs), 10-30 ng/ml anti-CD3 mAb (e.g., clone OKT3 or SPVT3b). The cells are cultured in a humidified incubator at 37° C. with 5% CO₂, and monitored visually every 1-2 days for evidence of acidification of the culture medium and by light microscopy for signs of proliferation. When acidification becomes apparent (yellow color) 25-50% of the culture supernatant is replaced with fresh medium, and the cells are resuspended, diluted with fresh medium, and split into new wells whenever sufficient proliferation has occurred. Once sufficient iNKT cell expansion has occurred to permit analysis without compromising the growth of the culture (typically after 2-3 weeks), about 5-10×10⁴ cells are removed and tested by flow cytometry to establish the purity of the culture using antibodies against CD3, CD4, and either lipid-loaded CD tetramer reagent or an antibody (clone 6B11) that is specific for the T cell receptor of iNKT cells. If necessary, the culture can be further purified by flow cytometric or magnetic sorting to remove contaminating cells (typically only cultures containing ≥99% CD4⁺ iNKT cells are used for generating conjugates). After sufficient expansion of the iNKT cells has occurred and the rate of proliferation has slowed to doubling approximately every 3-4 days (typically after 4-6 weeks of culture), iNKT cells are frozen in aliquots at a concentration of 5-20×10⁶ cells/ml using CryoStor CS10 freezing medium (StemCell Technologies).

Generation of monocyte-derived dendritic cells: Peripheral blood (5-50 ml) is collected into a heparinized tube (10-30 USP heparin/ml blood). Notably, in most cases this sample would be collected from the person who would eventually receive the iNKT+DC conjugate immunotherapy, though in certain situations (e.g., patients who have received a hematopoietic stem cell transplant), the DC donor might be a different person. PBMCs are isolated by density gradient centrifugation, and monocytes are isolated by positive-selection magnetic sorting using anti-CD14 coated beads (Miltenyi Corp. or StemCell Technologies) according to the manufacturer's protocol. The purified CD14⁺ monocytes are suspended in culture medium prepared according to the following recipe: RPMI 1640 medium, 10% fetal bovine serum, 100 IU/ml Penicillin, 100 μg/ml Streptomycin, 300 U/ml recombinant human GM-CSF, 200 U/ml recombinant human IL-4. The cells are added to sterile polystyrene tissue culture plates at 0.5-1×10⁶ cells/ml and cultured in a humidified incubator at 37° C. with 5% CO₂. Differentiation into a dendritic cell (DC) phenotype typically starts to become apparent within 2 days of culture, although we typically generate conjugates from cells that have been incubated in differentiation medium for 3 days. Successful differentiation and purity of the resulting culture is verified by flow cytometric analysis using fluorescently-labeled antibodies against CD14, CD209 (DC-SIGN), CD83, CD3, CD19, CD56. Immature DCs are identified as cells expressing CD209, with little or no detectable CD14, and lacking the other markers. If necessary, cultures can be further purified by magnetic or flow cytometric sorting.

Generation of iNKT+DC conjugates: iNKT cells are thawed 1-2 days prior to conjugation, and cultured in the iNKT cell growth medium described above. DCs are exposed to a source of relevant antigen. iNKT cells and DCs are mixed at a ratio of 1-5 iNKT cells per DC, suspended in RPMI 1640 medium containing 10% fetal bovine serum, 100 IU/ml Penicillin and 100 μg/ml Streptomycin, and co-cultured for 1-24 hours in a humidified incubator at 37° C. with 5% CO₂. In most of the experiments disclosed herein, 24 hours of co-culture was used to generate the conjugates. However, we have observed that conjugate formation occurs within 1 hour and conjugates appear to persist in the cultures for at least 96 hours. Flow cytometric analysis is performed to validate iNKT+DC conjugate formation and up-regulation of key co-stimulatory molecules (e.g., CD70, CD80, CD86). In these experiments, we have used the unseparated iNKT+DC mixture to assess efficacy. However, we envision that for the purposes of generating the immunotherapy reagent the iNKT+DC conjugates would be isolated by flow cytometric sorting prior to use.

What is claimed:

1. A stable in vitro-derived multicell conjugate between an invariant natural killer T (iNKT) cell and a dendritic cell (DC).

2. The stable multicell conjugate of claim 1, wherein the conjugate is maintained in culture for at least 30 minutes.

3. The stable multicell conjugate of claim 1, the conjugate being maintained in culture for at least 24 hours.

4. The stable multicell conjugate of claim 1, wherein the iNKT cell within the conjugate:
(a) expresses an appropriately rearranged TCR;
(b) expresses one or more markers selected from the group consisting of CD4, and CD3; or
(c) both (a) and (b).

5. The stable multicell conjugate of claim 1, wherein the conjugate is maintained in culture for at least 96 hours.

6. The stable multicell conjugate of claim 1, wherein the DC expresses major histocompatibility complex molecules I or II (WIC I or MEW II).

7. The stable multicell conjugate of claim 1, wherein the DC is allogenic to the iNKT cell or autologous to the iNKT cell.

8. The stable multicell conjugate of claim 1, wherein the multicell conjugate secretes IL-12p70, INF-γ or both into the culture medium.

9. The stable multicell conjugate of claim 1, wherein the DC cell expresses one or more of the molecules selected from CD80, CD83, CD86, CD134L (OX40L), CD137L (41BBL), CD215 (IL-15Rα), and combinations thereof on its surface.

10. The stable multicell conjugate of claim 9, wherein the DC within the conjugate expresses one or more co-stimulatory molecules for at least 24 hours in vitro.

11. The stable multicell conjugate claim 1, wherein the iNKT cell expresses CD70 on its surface after conjugation.

12. The stable multicell conjugate of claim 1, wherein the multicell conjugate comprises two or three cells per conjugate.

13. The stable multicell conjugate of claim 1, wherein the DCs has loaded with an antigen.

14. The stable multicell conjugate of claim 13, wherein the antigen is selected from a tumor antigen, a pathogenic antigen and a superantigen.

15. A composition comprising the stable multicell conjugate of claim 1 and a pharmaceutically acceptable carrier.

16. A method of producing a multicell conjugate between an invariant natural killer (iNK) T cell and a dendritic cell (DC), the method comprising:
co-culturing an iNKT cell and a DC for a sufficient amount of time to form a stable multicell conjugate.

17. The method of claim 16, wherein the iNKT cell and DC are co-cultured at an iNKT:DC ratio of 1:1 to about 5:1.

18. The method of claim 16, wherein the DC is contacted with lipidated human serum albumin or a cytokine prior to co-culturing, wherein the contacting increases the ability of the DC to form DC-iNKT conjugates in in vitro culture.

19. The method of claim 16, wherein the dendritic cell is obtained from a monocyte by a method comprising:
(i) culturing a monocyte in culture media comprising GM-CSF and IL-4 for a sufficient time to differentiate the monocyte into a DC.

20. The method of claim 16, the method further comprising:
(ii) isolating the monocyte from a blood sample of a subject before differentiation, or
(iii) isolating the monocyte from a tissue of a subject.

21. The method of claim 16, wherein the DC is generated by:
(i) differentiating CD34⁺ hematopoietic progenitor cells in vitro from a bone marrow sample, G-CSF mobilized peripheral blood, or other tissues into DCs; or
(ii) differentiating induced pluripotent stem cells (iPSCs) into DCs.

22. The method of claim 16, wherein the DC are generated by:
a) differentiating from CD34⁺ hematopoietic progenitor cells or induced pluripotent stem cells into CD1a−/CD14⁺ monocytes in culture;
b) isolating the CD1a−/CD14⁺ monocytes of step (a); and
c) differentiating the isolated monocytes into DC by culturing in medium comprising GM-CSF and IL4 for a sufficient time, wherein DCs are produced that show elevated CD209 and reduced CD14.

23. The method of claim 21, wherein
(a) the CD34⁺ hematopoietic progenitor or iPSCs are allogeneic from the subject in need;
(b) the CD34⁺ hematopoietic progenitor or iPSCs are genetically modified to express a limited selection of MHC molecules;
(c) the CD34⁺ hematopoietic progenitor or IPSCs express MHC molecules to match the subject to be treated; or
(d) combinations of (a)-(c).

24. The method of claim 16, further comprising:
(a) contacting the DC in culture with an antigen prior to co-culturing with the iNKT cell;
(b) concurrently contacting the DC with an antigen and co-culturing with the iNKT cell; or
(c) contacting the iNKT+DC conjugate with the antigen subsequent to conjugate formation.

25. A method of treating a subject with a condition associated with an antigen, the method comprising administering an effective amount of the multicell conjugate of claim 1 to treat the condition.

26. A method of activating T cells in a subject in need thereof, the method comprising administering an effective amount of the stable multicell conjugate of claim 1 to the subject, wherein T cell are activated.

27. A method of activating an immune response against an antigen within a subject, the method comprising: administering an effective amount of the multicell conjugate of claim 1 to the subject, wherein an immune response is activated against the antigen in the subject.
